SEQUENCE LISTING (4 sequences)

SEQ ID NO: 1 - protein, 141 aa, Artificial Sequence
  Synthetic
TYKITVRVYQTNPDAFFHPVEKTVWKYANGGTWTITDDQHVLTMGGSGTSGTLRFHADNGESFTATFGVHNYKRWCDIVTNLAADETGMVINQQYYSQKNREEARERQLSNYQVKNAKGRNFQIVYTEAEGNDLHANLIIG

SEQ ID NO: 2 - protein, 141 aa, Artificial Sequence
  Synthetic
VYKITVRVYQTNPDAFFHPVEKTVWKYANGGTWSITDDQHVLTMGGSGTSGTLRFHADNGESFTATFGVHNYKRWCDIVTNLAADETGMVINQQYYSQKNREEARERQLSNYQVKNAKGRNFQIVYTEAEGNDLHANLIIG

SEQ ID NO: 3 - protein, 141 aa, Artificial Sequence
  Synthetic
VYKITVRVYQTNPDAFFHPVEKTVWKYADGGTWSITDDQHVLTMGGSGTSGTLRFHADNGESFTATFGVHDYKRWCDIVTDLAADETGMVINQEYYSEKDREEARERQNSNYEVKDAKGRNFEIVYTEAEGNDLHADLIIG

SEQ ID NO: 4 - protein, 141 aa, Artificial Sequence
  Synthetic
TYKITVRVYQTNPNAFFHPVEKTVWKYANGGTWTITDDQHVLTMGGSGTSGTLRFHADNGESFTATFGVHNYKRWCDIVTNLAADETGMVINQQYYSQKNREEARERQLSNYEVKNAKGRNFEIVYTEAEGNDLHANLIIG

(19) United States
(12) Patent Application Publication
(10) Pub. No.: US 2024/0398899 A1
(43) Pub. Date: Dec. 5, 2024

(54) LECTIN PROTEIN FOR TREATMENT AND PREVENTION OF NEURODEGENERATIVE DISEASES

(71) Applicant: Unichem Laboratories Limited, Mumbai, Maharashtra (IN)

(72) Inventors: Sarvanakumar Iyappan, Hyderabad (IN); Dilip Pawar, West Mumbai (IN); Dhananjay Sathe, Thane (IN)

(21) Appl. No.: 18/260,404

(22) PCT Filed: Jan. 5, 2022
(86) PCT No.: PCT/IB2022/050056

§ 371 Date: Jul. 5, 2023

(30) Foreign Application Priority Data
Jan. 7, 2021 (IN) 202121000832

(51) Int. Cl. A61K 38/17; A61P 25/16; A61P 25/28; C07K 14/37

(52) U.S. Cl. CPC A61K 38/178; A61P 25/16; A61P 25/28; C07K 14/37

(57) ABSTRACT

The present invention relates to the lectin protein for the treatment and prevention of neurodegenerative diseases. The invention further relates to the recombinant lectin protein is derived from Sclerotium rolfsii lectin having sequence 60% homologous to SEQ ID NO: 4 for the treatment and prevention of Neurodegenerative diseases. The invention specifically relates to lectin protein and its variants is derived from Sclerotium rolfsii lectin having sequence 60% homologous to SEQ ID NO: 4 for the treatment of prevention of Parkinson's disease, Alzheimer's disease, Dementia and symptoms of dementia.

### FIELD OF INVENTION

The present invention relates to the protein for the treatment and prevention of neurodegenerative disease.

### BACKGROUND

Neurodegenerative disease is a condition which causes progressive irreversible damage to the neural system. As a consequence of this, neurodegenerative disease results either in ataxia or Dementia. The pathogenesis of the Neurodegenerative diseases is characterized by extracellular and intercellular deposition of the amyloid beta (AB) peptides and hyper-phosphorylation of Tau protein resulting in plagues and neurofibrillary tangle respectively, leads to oxidative stress and result in neuro inflammation; Post-translational modification of α-synuclein, such as phosphorylation, ubiquitination and nitration, has been widely implicated in α-synuclein aggregation process that leads to Lewy body formation and death of dopamine neurons, In addition to this, any abnormality in any one of the pathways comprising: intracellular mechanism (for example apoptosis, autophagy, mitochondrial dysfunction, oxidative DNA damage and repair, ubiquitin-proteasome system); local tissue environment (such as cell adhesion, endocytosis, neurotransmission, prions and transmissible factors); systemic environment (inflammation and immune dysfunction, lipid, metabolic endocrine factors vascular changes) and development and aging (for example epigenetic changes, neurotrophic factors, telomeres) etc. cause neurodegerative disease.

Dementia is defined as cognitive impairment in more than one cognitive area characterized by loss of intellectual ability of sufficient severity to interfere either with occupational functioning, usual social activities or relationship of a person in the absence of gross clouding of consciousness or with motor involvement (Sharma and Singh et al., 2010 Indian journal of Pharmacology Vol: 42, issue: 3; Page 164-167). Cognitive areas involved in dementia includes language (aphasia), motor (apraxia), agnosia (failure in recognition) & executive functions (abstract reasoning, judgment and planning) (Parris M. Kidd; Alternative Medicine Review. 2008, Vol. 13 Issue 2, p85-115. 31p.) There are many types of Dementia, including Alzheimer's disease, vascular dementia, and dementia with Lewy body, frontotemporal dementia, and dementia associated with Parkinson's disease, Huntington's disease, dementia due to hydrocephalus, Wernicke Korsakoff syndrome, and Creutzfeldt-Jakob disease dementias (Husband, A. and Worsley, A 2006, The Pharmaceutical Journal, 277 (7426). pp. 579-582).

Our aging society is confronted with a remarkable increase in incidences of age-related neurodegenerative diseases (Rasalan and kee 2013; Genes & Genomics volume 35, pages 425-440). Therapeutic and non-therapeutic approaches such as change or intervention in life style or proper diet (Gurjit et al., 2019 Front Aging Neurosci. 2019; 11:369) are the recourses available for the treatment of neurodegenerative diseases. In therapeutic approaches, commonly used and approved therapeutic agents for the treatment or prevention of neurodegenerative diseases are cholinesterase inhibitor (Galantamine, donepezil, rivastigmine), memantine, istradefylline, dopamine agonists (pramipexole, apomorphine), levodopa/carbidopa, monoclonal antibodies such as daclizumab, natalizumab, alemtuzumab and immunomodulators such as teriflunomide. These medicines cause severe side effects such as convulsions, nausea, dizziness, bradycardia, fall and even death.

Hence, there is an exigency to develop new potent therapeutics, which have intrinsic property to alleviate learning and memory impairments and restore normal expression level of nerve growth factor (NGF) and Acetylcholine esterase (AChE) to exert a protective effect on brain against disease such as Alzheimer's and Parkinson's.

The lectins are carbohydrate binding protein, which are ubiquitous in plant, animals and microorganisms. Agglutinating property of the lectin elevated its application in advance medical research. The therapeutic potential of lectin in neurodegenerative disease has also been well studied.

U.S. application No. 20200017578 discloses use of lectin with binding specificity for a sialic acid such as Limax flavus agglutinin (LFA), Limulus polyphemus agglutinin (LPA), Paecilomyes japonica agglutinin (PJA), lobster agglutinin I, or Penaeus monodin lectin for the treatment of neurodegenerative disorder such as Alzheimer's Disease.

U.S. Pat. No. 8,916,387 describes a method for the prevention, treatment and diagnosis of Alzheimer's disease, based on the glycosylation pattern of amyloid-beta peptides in body fluids and tissues. Lectins from mistletoe, Maackia amurensis and Agrocybe cylindracea are disclosed as useful as medicine or diagnostic agent for prevention and treatment of cortical atrophy, neuronal loss, region-specific amyloid deposition, neuritic plaques and neurofibrillary tangles. It is further disclosed that the lectins are used to treat or prevent a disease where amyloid beta plaque deposition is implicated, wherein the disease is selected from the group consisting of cerebral amyloid angiopathy and Alzheimer's disease or HIV associated neurocognitive diseases.

Tetranectin is a human homotrimeric 21 KD protein belonging to the C-type lectin family. It is found that the level of tetranectin in cerebrospinal fluid decreased dramatically in patients with Parkinson's disease compared with normal control subjects and tetranectin acts as a neuroprotective agent by inhibiting apoptosis and autophagy in 1-methyl-4-phenylpyridine-induced neurotoxicity (Qiang Xie et. al, 2018 World Neurosurgery Volume 122, Pages e375-e382).

The treatment of neurodegenerative disease using agents such as synthetic drugs or peptides is well established in prior arts, wherein lectin is used as a cell surface binding agent or delivery agent. There is very limited information on the use of lectins as a therapeutic agent in the treatment and prevention of neurodegenerative diseases. Majorly in the above studies the following lectins have been explored for the treatment or prevention of neurological diseases: Plant lectins such as Maackia amurensis, animal lectin such as Penaeus monodin-Limax flavus (garden slug); Limulus polyphemus and Fungal lectin Agrocybe cylindracea.

The use of some indigenous lectins as a binding or delivery agent for pharmaceutical active agents is known and has been reported for the treatment of neurodegenerative disease as well (e.g. US20070243132). However, the potency of lectin as a therapeutic agent in the treatment or prevention of neurodegenerative disease is not studied in detail. Thus, there is a need for the exploration and identification of a potent lectin that alleviate the brain cognitive impairment and restore the expression level of neurotrophic factors and cholinesterase and confers high therapeutic efficacy against neurodegenerative disease.

Sclerotium rolfsii lectin (SRL) is a lectin that has been isolated from the sclerotial bodies of the soil-borne phytopathogenic fungus S. rolfsii. SRL has specificity towards Thomsen-Friedenreich (TF) antigen and Tn antigen. TF antigen is a disaccharide (Galβ1→3GalNAc-α-Ser/Thr) that is overexpressed on the cell surface of various human cancer cells. Tn antigen is a monosaccharide (GalNAc-α-Ser/Thr). WO 2010/095143 discloses recombinant lectin variants Rec-2 and Rec-3, which are derived from the native SRL sequence by the substitution of 3 or 5 amino acids respectively. The crystal structure of these variants has been reported (Peppa et al., Molecules. Jun. 12, 2015; 20 (6): 10848-65). WO 2014/203261 discloses a recombinant lectin variant derived from the native SRL sequence by the substitution of 12 amino acids.

### OBJECT OF THE INVENTION

The object of the present invention is to develop a new method for prevention and treatment of Neurodegenerative disease. The new method signifies a method comprising new therapeutically effective agent for the prevention and treatment of neurodegenerative diseases. Accordingly, it is an object to establish the use of new therapeutic agent in the method of treatment and prevention of neurodegenerative disease, wherein the new therapeutic agent is a recombinant lectin.

Another object of the invention is to provide a recombinant lectin for the treatment and prevention of Neurodegenerative disease. Accordingly, the object is to provide recombinant lectin for the treatment and prevention of diseases causing dementia. The object is also to particularly provide a recombinant lectin for the treatment and prevention of Alzheimer's and Parkinson's disease.

Yet another object of the invention is to provide a composition comprising recombinant lectin for the treatment and prevention of neurodegenerative disease.

The use of composition comprising recombinant lectin for the treatment and prevention of neurodegenerative disease is also an object of this invention.

### SUMMARY OF THE INVENTION

The present invention relates to a recombinant lectin protein for the treatment or prevention of neurodegenerative disease wherein the recombinant lectin protein is derived from Sclerotium rolfsii lectin.

The present invention further relates to a pharmaceutical composition for the treatment or prevention of neurodegenerative disease comprising therapeutically effective amount of recombinant lectin protein derived from Sclerotium rolfsii lectin and a pharmaceutically acceptable excipient.

The present invention also relates to a method for the treatment or prevention of neurodegenerative disease wherein the method comprises administration of an effective amount of recombinant lectin protein derived from Sclerotium rolfsii lectin to the subject.

The present invention relates to the use of recombinant lectin protein derived from Sclerotium rolfsii lectin for the treatment or prevention of Neurodegenerative disease.

In yet another aspect, the present invention relates to a recombinant lectin protein for the treatment or prevention of neurodegenerative disease in a subject, wherein the recombinant lectin protein is derived from Sclerotium rolfsii lectin, and wherein the neurodegenerative disease is selected from Alzheimer's disease, Parkinson's disease, dementia, cognitive disorder and symptoms related to dementia.

In yet another aspect, the present invention relates to a recombinant lectin protein for the treatment or prevention of neurodegenerative disease in a subject, wherein the recombinant lectin protein is derived from Sclerotium rolfsii lectin, and wherein the neurodegenerative disease is selected from Huntington's disease, prion diseases such as Creutzfeld-Jacob disease, Lewy Body disease, diffuse Lewy body disease (DLBD), polyglutamine (polyQ)-repeat diseases, cerebral degenerative diseases, spinal and bulbar muscular atrophy (SBMA), Ataxia, Pick's disease, primary progressive aphasia, multiple system atrophy, pantothenate kinase-associated neurodegeneration (PANK), spinal degenerative disease/motor neuron degenerative diseases, hippocampal sclerosis, corticobasal degeneration, Batten disease

In yet another aspect, the present invention relates to a recombinant lectin protein for the treatment or prevention of neurodegenerative disease in a subject, wherein the recombinant lectin protein is derived from Sclerotium rolfsii lectin, and wherein the neurodegenerative disease is selected from motor neuron disease like Amyotrophic lateral sclerosis (ALS, also termed Lou Gehrig's disease), primary lateral sclerosis (PLS), progressive bulbar palsy (PBP) a variant of ALS, Pseudo bulbar palsy and Hereditary spastic paraplegia.

According to yet another aspect, the present invention provides a method for the treatment or prevention of neurodegenerative disease in a subject, wherein the method comprises administration of an effective amount of recombinant lectin protein derived from Sclerotium rolfsii lectin to the subject, wherein the neurodegenerative disease is selected from Alzheimer's disease, Parkinson's disease, dementia, cognitive disorder and symptoms related to dementia.

In yet another aspect, the present invention provides a method for the treatment or prevention of neurodegenerative disease in a subject, wherein the method comprises administration of an effective amount of recombinant lectin protein derived from Sclerotium rolfsii lectin to the subject, wherein the neurodegenerative disease is selected from Huntington's disease, prion diseases such as Creutzfeld-Jacob disease, Lewy Body disease, diffuse Lewy body disease (DLBD), polyglutamine (polyQ)-repeat diseases, cerebral degenerative diseases, spinal and bulbar muscular atrophy (SBMA), Ataxia, Pick's disease, primary progressive aphasia, multiple system atrophy, pantothenate kinase-associated neurodegeneration (PANK), spinal degenerative disease/motor neuron degenerative diseases, hippocampal sclerosis, corticobasal degeneration, Batten disease

In yet another aspect, the present invention provides a method for the treatment or prevention of neurodegenerative disease in a subject, wherein the method comprises administration of an effective amount of recombinant lectin protein derived from Sclerotium rolfsii lectin to the subject, wherein the neurodegenerative disease is selected from motor neuron disease like Amyotrophic lateral sclerosis (ALS, also termed Lou Gehrig's disease), primary lateral sclerosis (PLS), progressive bulbar palsy (PBP) a variant of ALS, Pseudo bulbar palsy and Hereditary spastic paraplegia.

According to another aspect of the invention, there is provided a pharmaceutical composition for the treatment or prevention of neurodegenerative disease comprising therapeutically effective amount of recombinant lectin protein derived from Sclerotium rolfsii lectin and a pharmaceutically acceptable excipient, wherein the neurodegenerative disease is selected from Alzheimer's disease, Parkinson's disease, dementia, cognitive disorder and symptoms related to dementia.

In yet another aspect, the present invention provides a pharmaceutical composition for the treatment or prevention of neurodegenerative disease comprising therapeutically effective amount of recombinant lectin protein derived from Sclerotium rolfsii lectin and a pharmaceutically acceptable excipient, wherein the neurodegenerative disease is selected from Huntington's disease, prion diseases such as Creutzfeld-Jacob disease, Lewy Body disease, diffuse Lewy body disease (DLBD), polyglutamine (polyQ)-repeat diseases, cerebral degenerative diseases, spinal and bulbar muscular atrophy (SBMA), Ataxia, Pick's disease, primary progressive aphasia, multiple system atrophy, pantothenate kinase-associated neurodegeneration (PANK), spinal degenerative disease/motor neuron degenerative diseases, hippocampal sclerosis, corticobasal degeneration, Batten disease

In yet another aspect, the present invention provides a pharmaceutical composition for the treatment or prevention of neurodegenerative disease comprising therapeutically effective amount of recombinant lectin protein derived from Sclerotium rolfsii lectin and a pharmaceutically acceptable excipient, wherein the neurodegenerative disease is selected from motor neuron disease like Amyotrophic lateral sclerosis (ALS, also termed Lou Gehrig's disease), primary lateral sclerosis (PLS), progressive bulbar palsy (PBP) a variant of ALS, Pseudo bulbar palsy and Hereditary spastic paraplegia,

According to yet another aspect of the invention, there is provided the use of recombinant lectin protein for the treatment or prevention of neurodegenerative disease in a subject, wherein the recombinant lectin protein is derived from Sclerotium rolfsii lectin, and wherein the neurodegenerative disease is selected from Alzheimer's disease, Parkinson's disease, dementia, cognitive disorder and symptoms related to dementia.

In yet another aspect, there is provided the use of recombinant lectin protein for the treatment or prevention of neurodegenerative disease in a subject, wherein the recombinant lectin protein is derived from Sclerotium rolfsii lectin, and wherein the neurodegenerative disease is selected from Huntington's disease, prion diseases such as Creutzfeld-Jacob disease, Lewy Body disease, diffuse Lewy body disease (DLBD), polyglutamine (polyQ)-repeat diseases, cerebral degenerative diseases, spinal and bulbar muscular atrophy (SBMA), Ataxia, Pick's disease, primary progressive aphasia, multiple system atrophy, pantothenate kinase-associated neurodegeneration (PANK), spinal degenerative disease/motor neuron degenerative diseases, hippocampal sclerosis, corticobasal degeneration, Batten disease

In yet another aspect, there is provided the use of recombinant lectin protein for the treatment or prevention of neurodegenerative disease in a subject, wherein the recombinant lectin protein is derived from Sclerotium rolfsii lectin, and wherein the neurodegenerative disease is selected from motor neuron disease like Amyotrophic lateral sclerosis (ALS, also termed Lou Gehrig's disease), primary lateral sclerosis (PLS), progressive bulbar palsy (PBP) a variant of ALS, Pseudo bulbar palsy and Hereditary spastic paraplegia,

According to an aspect of the invention, there is provided a method for inducing neuronal outgrowth, wherein the method comprises administration of an effective amount of recombinant lectin protein derived from Sclerotium rolfsii lectin to the subject.

According to the preceding aspects of invention, the recombinant lectin protein comprises an amino acid sequence selected from
i) SEQ ID NO. 4, or
ii) an amino acid sequence having atleast 70% identity to SEQ ID NO 4.

According to the preceding aspects of the invention, recombinant lectin protein comprises an amino acid sequence having atleast 70%, 75%, 80%, 85%, 90%, 91%, 92%, 93%, 94%, 95%, 96%, 97%, 98%, 99% identity to SEQ ID NO. 4.

According to the preceding aspects of the invention, the effective amount recombinant lectin protein administered for the treatment or prevention of the neurodegenerative disease is in the range from 0.01 mg/kg to 1000 mg/kg body weight of the subject.

According to one particular aspect of the invention, there is provided a recombinant lectin protein having sequence of SEQ ID NO: 1, SEQ ID NO: 2, SEQ ID NO: 3 or SEQ ID NO: 4 for the treatment or prevention of neurodegenerative disease.

According to another particular aspect of the invention, there is provided a method of treatment or prevention of neurodegenerative disease, wherein the method comprises administration of an effective amount of recombinant lectin protein having sequence of SEQ ID NO: 1, SEQ ID NO: 2, SEQ ID NO: 3, or SEQ ID NO: 4 to the subject.

According to yet another particular aspect of the invention, there is provided a composition for the treatment or prevention of neurodegenerative disease in a subject, wherein the composition comprises therapeutically effective amount of recombinant lectin protein having sequence of SEQ ID NO: 1, SEQ ID NO: 2, SEQ ID NO: 3 or SEQ ID NO: 4.

According to another particular aspect of the present invention, there is provided the use of recombinant lectin protein having sequence of SEQ ID NO: 1, SEQ ID NO: 2, SEQ ID NO: 3, or SEQ ID NO: 4 for the treatment or prevention of neurodegenerative disease in a subject.

According to yet another particular aspect of the present invention, there is provided the use of composition comprising therapeutically effective amount recombinant lectin protein having sequence of SEQ ID NO: 1, SEQ ID NO: 2, SEQ ID NO: 3 or SEQ ID NO: 4 for the treatment or prevention of neurodegenerative disease in a subject.

In a particular aspect of the present invention, there is provided a recombinant lectin protein having sequence of SEQ ID NO: 1, SEQ ID NO: 2, SEQ ID NO: 3 or SEQ ID NO: 4 for the treatment or prevention of neurodegenerative disease in a subject, wherein the neurodegenerative disease is selected from Alzheimer's disease, Parkinson's disease, dementia, cognitive disorder and symptoms related to dementia.

In another particular aspect of the present invention, there is provided a method for the treatment or prevention of neurodegenerative disease in a subject, wherein the method comprises administration of an effective amount of recombinant lectin protein having sequence of SEQ ID NO: 1, SEQ ID NO: 2, SEQ ID NO: 3 or SEQ ID NO: 4 to the subject, wherein the neurodegenerative disease is selected from Alzheimer's disease, Parkinson's disease, dementia, cognitive disorder and symptoms related to dementia.

In yet another particular aspect of the present invention, there is provided a pharmaceutical composition for the treatment or prevention of neurodegenerative disease comprising therapeutically effective amount of recombinant lectin protein having sequence of SEQ ID NO: 1, SEQ ID NO: 2, SEQ ID NO: 3 or SEQ ID NO: 4 and a pharmaceutically acceptable excipient, wherein the neurodegenerative disease is selected from Alzheimer's disease, Parkinson's disease, dementia, cognitive disorder and symptoms related to dementia.

In yet another particular aspect of the present invention, there is provided the use of recombinant lectin protein having sequence of SEQ ID NO: 1, SEQ ID NO: 2, SEQ ID NO: 3 or SEQ ID NO: 4 for the treatment or prevention of neurodegenerative disease in a subject, wherein the neurodegenerative disease is selected from Alzheimer's disease, Parkinson's disease, dementia, cognitive disorder and symptoms related to dementia.

In an aspect of the present invention, there is provided a method for inducing neuronal outgrowth, wherein the method comprises administration of an effective amount of recombinant lectin protein having sequence of SEQ ID NO: 1, SEQ ID NO: 2, SEQ ID NO: 3 or SEQ ID NO: 4.

### Definition

The term “lectin” as used herein refers to a carbohydrate-binding protein.

The term “protein” as used herein refers to a polymer of amino acid residues.

The term “amino acid” as used herein refers to naturally occurring and synthetic amino acids, as well as amino acid analogues and amino acid mimetics that have a function that is similar to the naturally occurring amino acids. Naturally occurring amino acids are those encoded by the genetic code and include the proteinogenic amino acids. Naturally occurring amino acids also include those modified after translation in cells. Synthetic amino acids include non-canonical amino acids such as selenocysteine and pyrrolysine. Typically, synthetic amino acids are not proteinogenic amino acids.

The term “neural” or “Neural cells” are cells that reside in the brain, central and peripheral nerve systems, including, but not limited to, nerve cells, glial cell, oligodendrocyte, microglia cells or neural stem cells.

The terms disease or disorder are used interchangeably unless otherwise mentioned. A “disease” is a state of health of an animal wherein the animal cannot maintain homeostasis, and wherein if the disease is not ameliorated then the animal's health continues to deteriorate. “Disorder” in an animal is a state of health in which the animal is able to maintain homeostasis, but in which the animal's state of health is less favorable than it would be in the absence of the disorder. The term “disease” or “disorder” is used interchangeably, may also refers to any alteration in state of the body or of some of the organs, interrupting or disturbing the performance of the functions and/or causing symptoms such as discomfort, dysfunction, distress, or even death to the person afflicted or those in contact with a person. A disease or disorder can also relate to a distemper, ailing, ailment, malady, disorder, sickness, illness, complaint, inder-disposion or affectation.

The term “neurodegenerative disease” includes both neurodegenerative disease or disorder and is defined as the gradual and progressive loss of function or structure of neural tissue and/or neural tissue function. This leads to increase in impairment even death of neuronal cells. Probable causes of this disease or disorder may be ageing, genetic aberration or exposure to toxins, chemicals or viruses. Sometimes the cause may be a medical condition such as alcoholism, a tumor, or a stroke. Usually neurodegenerative diseases cause problems with body activities such as movement (called ataxias), mental functioning (called dementias), balancing, talking and breathing. In certain case it may also affect the heart functioning. A neurodegenerative disease according to the invention may include, but is not limited to, conditions where neurons are dysfunctional and/or degenerating. Non-limiting examples of such diseases are Alzheimer's disease (AD), Parkinson's disease (PD), Huntington's disease, dementia or symptoms of dementia that cause more than one neurodegenerative disease listed here, frontotemporal dementia (FTD), FTD caused by mutations in the progranulin protein or tau protein {e.g., progranulin-deficient FTLD), frontotemporal dementia with inclusion body myopathy (IBMPFD), frontotemporal dementia with motor neuron disease, Amyotrophic Lateral Sclerosis (ALS, also termed Lou Gehrig's disease), amyotrophic lateral sclerosis with dementia (ALSD), primary lateral sclerosis (PLS), spinal muscular atrophy (SMA), Multiple Sclerosis (MS), prion diseases such as Creutzfeld-Jacob disease, Lewy Body disease, diffuse Lewy body disease (DLBD), polyglutamine (polyQ)-repeat diseases, trinucleotide repeat diseases, cerebral degenerative diseases, presenile dementia, senile dementia, Parkinsonism linked to chromosome 17 (FTDP-17), progressive supranuclear palsy (PSP), progressive bulbar palsy (PBP), psuedobulbar palsy, spinal and bulbar muscular atrophy (SBMA), Friedreich's ataxia, cerebellar ataxia, Pick's disease, primary progressive aphasia, corticobasal dementia, HIV-associated dementia, Parkinson's disease with dementia, dementia with Lewy bodies, multiple system atrophy, spinal muscular atrophy such as Werdnig-Hoffmann disease, Kugelberg-Welander disease or congenital SMA with arthrogryposis, progressive spinobulbar muscular atrophy such as Kennedy disease, spinocerebellar ataxia, pantothenate kinase-associated neurodegeneration (PANK), spinal degenerative disease/motor neuron degenerative diseases, upper motor neuron disorder, lower motor neuron disorder, Hallervorden-Spatz syndrome, amyotrophic lateral sclerosis-parkinsonism-dementia, Guam-Parkinsonism dementia, hippocampal sclerosis, corticobasal degeneration, Alexander disease, Apler's disease, Krabbe's disease, neuroborreliosis, neurosyphilis, Sandhoff disease, Schilder's disease, Batten disease, Cockayne syndrome, Kearns-Sayre syndrome, Gerstmann-Straussler-Scheinker syndrome, hereditary spastic paraparesis, Leigh's syndrome, demyelinating diseases, other brain disorders such as bipolar disorder, epilepsy, schizophrenia, depression, mania, autism, ADHD, brain trauma injuries and stroke.

It is well understood by the skilled person that the ‘neurodegenerative disease’ listed above, in a very broad sense may be categorized as “Dementia” which for example includes diseases such as Alzheimer's disease, Frontotemporal dementia (picks disease), Lewy body dementia, neurofibrillary tangle dementia, and symptoms related to dementia, Creutzfeld-Jacob disease (which has similar clinical manifestation as that of Alzheimer), Hippocampal sclerosis, Schilder's disease, Vascular (Multi-infarct) dementia, Huntington, Parkinson's disease associated with AD, diffused Lewy body disease (DLBD); ‘Parkinson's disease and Parkinson like disease’ such as progressive supranuclear palsy (PSP), multiple system atrophy (MSA), and corticobasal degeneration (CBD); ‘Motor neuron disease’ may include diseases affecting upper/lower motor neuron regions such as for example, amyotrophic lateral sclerosis (ALS), primary lateral sclerosis (PLS), and progressive bulbar palsy (PBP) a variant of ALS, pseudo bulbar palsy and hereditary spastic paraplegia. These categories may be based on the cause or mechanism of the disease, organ or body part or body function affected by these disease or the relationships between the two diseases.

The term “neuroprotective” or “cytoprotective is used interchangeably and refer to protection or prevention the neuronal cells from abnormalities caused by ageing, genetic aberration and external factors such as neurotoxin and restoration of the regular or normal functioning of the neurons.

Therapeutic agent or therapeutically effective agent as used interchangeably here, means an agent administered to a subject for reducing or abolishing one or more symptoms of the disease or disorder, wherein the agent according to the present invention is recombinant lectin and the disease or disorder is neurodegenerative disease.

“Neuronal Outgrowth” or “neurite” as used interchangeably here, means a projection from the cell body of a neuron including, e.g., an axon or a dendrite

The term “modulation” as used herein refers to alter or regulate the physiological mechanisms. (e.g.: membrane potential) of the organelle.

The term ‘therapeutically effective amount’ as used herein is an amount sufficient to effect desired therapeutic benefit, wherein therapeutic benefit implies effect in the treatment or prevention of neurodegenerative disease or diseases. The effect is such that the subject is either free from the disease or diseases or the symptoms of the disease or diseases are controlled or reduced or there is a delay in the onset or progress of the disease or diseases. A therapeutically effective amount can be administered in one or more administrations. For purposes of this invention, a therapeutically effective amount of a recombinant protein is an amount that is sufficient to palliate, ameliorate, stabilize, reverse, prevent, slow or delay the progression of the disease state.

The terms “homology” or “homologous” as used herein refer to two or more referenced entities that share at least partial identity over a given region or portion. Areas, regions or domains of homology or identity refer to a portion of two or more referenced entities that share homology or are the same. Thus, where two sequences are identical over one or more sequence regions, they share identity in these regions. Substantial homology refers to a molecule that is structurally or functionally conserved such that it has or is predicted to have at least partial structure or function of one or more of the structures or functions (e.g., a biological function or activity) of the reference molecule, or a relevant/corresponding region or portion of the reference molecule to which it shares homology.

In one embodiment, the percentage “homology” between two sequences is determined using the BLASTP algorithm with default parameters (Altschul et al. Nucleic Acids Res. Sep. 1, 1997; 25 (17): 3389-402). In particular, the BLAST algorithm can be accessed on the internet using the URL: https://blast.ncbi.nlm.nih.gov/Blast.cgi. In an alternative embodiment, for global sequence alignments, percentage homology between two sequences is determined using the EMBOSS Needle algorithm using default parameters. In particular, the EMBOSS Needle algorithm can be accessed on the internet using the URL: https://www.ebi.ac.uk/Tools/psa/emboss_needle/.

Unless otherwise indicated, the term “homology” is used interchangeably with the term “sequence identity” in the present specification.

### DESCRIPTION OF DRAWINGS AND TABLES

FIG. 1. Pictorial representation of effect of SEQ ID NO: 1 on neurite formation in neuronal cells (pc12) in basal model

FIG. 2. Pictorial representation of effect of SEQ ID NO: 1 on neurite formation in neuronal cells (pc12) against MPP+ induced damage

FIG. 3: Histopathology Images (H&E staining; 100×)

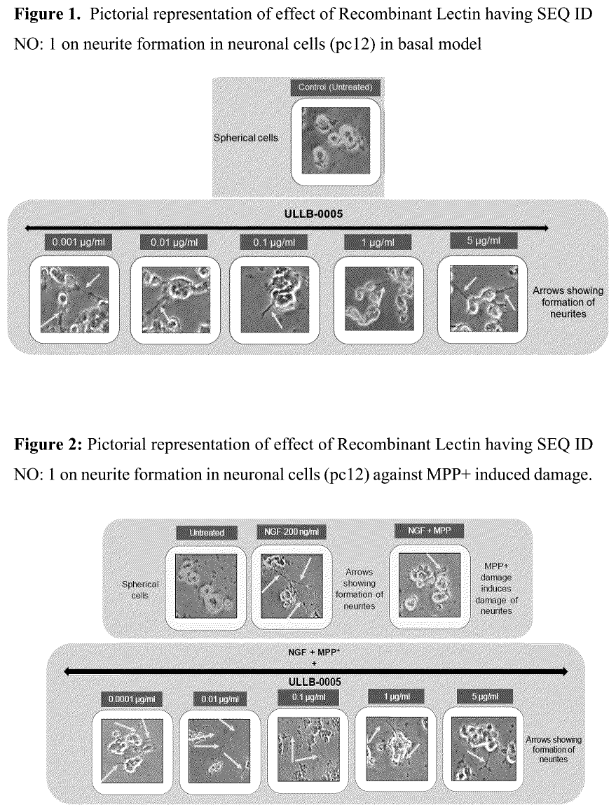

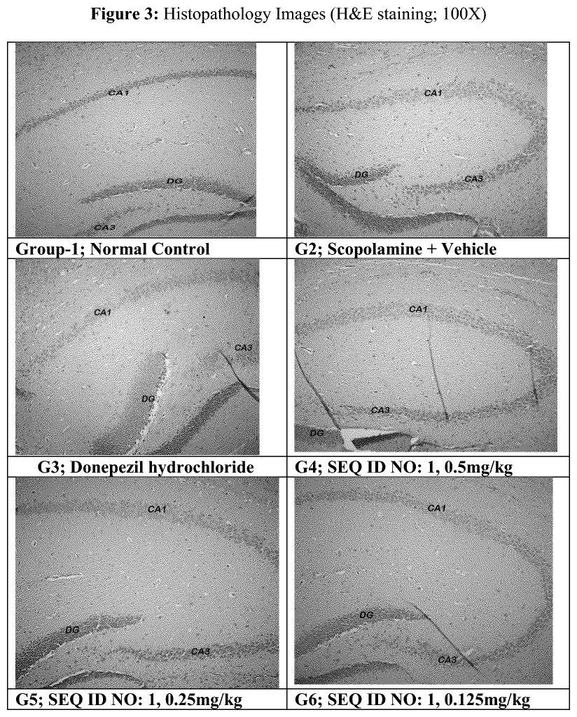

Table-1—Cytoprotective effect of SEQ ID NO: 1 in neuronal cells (SH-SY5Y) against neurotoxin (MPP+) induced damage

Table-2—Anti-apoptotic effect of SEQ ID NO: 1 in neuronal cells (SH-SY5Y) via restoration of mitochondrial membrane potential against MPP+ iodide damage

Table-3—Anti-apoptotic effect of SEQ ID NO: 1 in neuronal cells (SH-SY5Y) via decrease in Annexin positive cell population against MPP+ iodide damage

Table-4—Anti-apoptotic effect of SEQ ID NO: 1 in neuronal cells (SH-SY5Y) via decrease in Sub (G0/G1) cell population against MPP+ iodide induced damage

Table 5: Effect of SEQ ID NO: 1 on neurite formation in neuronal cells (pc12) in basal model

Table 6: Protective effect of SEQ ID NO: 1 on neurite formation in neuronal cells (pc12) against mpp+ induced damage

Table: 7 Effect of SEQ ID NO: 1 on expression of biomarkers associated with Alzheimer's disease in Neuronal cell line (SH-SY5Y)

Table: 8 Effect of SEQ ID NO: 1 on expression of biomarkers associated with Parkinson disease in Neuronal cell line (SH-SY5Y)

Table 9: Allocation of Animals

Table 10: Mean Transfer Latency Time (Sec.)

Table 11: Effect of SEQ ID NO: 1 on brain nerve growth factor (NGF) (pg/ml)

Table 12: Effect of SEQ ID NO: 1 on brain Acetylcholine esterase (mU/ml)

Table 13: Effect of SEQ ID NO: 1 on brain TNF-alpha (pg/ml)

Table 14: Histopathology (Mean score)

### Sequence Representation:

SEQ ID NO 4: represents the native S. rolfsii lectin amino acid sequence (reported as SEQ ID NO:1 in WO2010/095143), has the following sequence:

TYKITVRVYQTNPNAFFHPVEKTVWKYANGGTWTITDDQHVLTMGGSG
TSGTLRFHADNGESFTATFGVHNYKRWCDIVTNLAADETGMVINQQYYS
QKNREEARERQLSNYEVKNAKGRNFEIVYTEAEGNDLHANLIIG

SEQ ID NO. 1: represents a variant of the S. rolfsii lectin amino acid sequence (reported as Rec-2 in WO2010/095143) has the following sequence:

TYKITVRVYQTNPDAFFHPVEKTVWKYANGGTWTITDDQHVLTMGGSG
TSGTLRFHADNGESFTATFGVHNYKRWCDIVTNLAADETGMVINQQYYS
QKNREEARERQLSNYQVKNAKGRNFQIVYTEAEGNDLHANLIIG

SEQ ID NO. 2: represents a variant of the S. rolfsii lectin amino acid sequence (reported as Rec-3 in WO 2010/095143) has the following sequence:

VYKITVRVYQTNPDAFFHPVEKTVWKYANGGTWSITDDQHVLTMGGSG
TSGTLRFHADNGESFTATFGVHNYKRWCDIVTNLAADETGMVINQQYYS
QKNREEARERQLSNYQVKNAKGRNFQIVYTEAEGNDLHANLIIG

SEQ ID NO. 3: represents a variant of the S. rolfsii lectin amino acid sequence (reported in WO 2014/203261) has the following sequence:

VYKITVRVYQTNPDAFFHPVEKTVWKYADGGTWSITDDQHVLTMGGSG
TSGTLRFHADNGESFTATFGVHDYKRWCDIVTDLAADETGMVINQEYYS
EKDREEARERONSNYEVKDAKGRNFEIVYTEAEGNDLHADLIIG

### DETAILED DESCRIPTION OF THE INVENTION

In the first aspect, the present invention furnishes a recombinant lectin for the treatment or prevention of neurodegenerative diseases wherein the recombinant lectin protein is derived from sclerotium rolfsii (S. rolfsii) lectin.

In an embodiment of the present invention, the lectin is derived from the group consisting of, but not limited to, fungus and plants. In some embodiments the lectin is derived from a soil borne phytopathogenic fungus, such as S. rolfsii.

By “derived from” it will be understood that the lectin may be isolated from its native environment, or that the lectin comprises an amino acid sequence which is identical or similar to a native sequence.

The lectin may comprise an amino acid sequence having at least 60% 70%, 80%, 90%, 95%, 96%, 97%, 98% or 99% homology to a native sequence. The native lectin may be isolated from S. rolfsii.

The lectin may comprise an amino acid sequence having at least 60% homology to SEQ ID NO: 4. In some embodiments, the lectin may comprise an amino acid sequence having at least 60% homology to SEQ ID NO: 1, 2, or 3. In some embodiments, the amino acid sequence has at least 70%, 80%, 90%, 95%, 96%, 97%, 98% or 99% homology to SEQ ID NO: 4. In some embodiments, the amino acid sequence has at least 70%, 80%, 90%, 95%, 96%, 97%, 98% or 99% homology to SEQ ID NO: 1, 2, or 3.

SEQ ID NO: 1 has 98% homology with SEQ ID NO: 4. SEQ ID NO: 2 has 96% homology with SEQ ID NO: 4. SEQ ID NO: 3 has 91% homology with SEQ ID NO: 4.

According to any one of the preceding aspects, the recombinant lectin is a modified lectin protein (i.e. a recombinant lectin protein having at least one amino acid modification in the molecule, preferably in a carbohydrate binding sites) as defined in WO2020/044296 which is incorporated herein by reference.

According to some specific embodiments of the present invention, the lectin comprises an amino acid sequence selected from the group consisting of SEQ ID NO: 1, SEQ ID NO: 2 or SEQ ID NO: 3.

According to one specific aspect of embodiment of the present invention the lectin protein of the present invention is preferably synthesized using recombinant technology. The methods for preparing recombinant proteins will be well-known to those skilled in the art. In one embodiment, the cloned nucleotide sequences encode modified lectin proteins that are close to the native lectin amino acid sequence, but which provide alternative properties. Alternatively, the nucleotide sequences encoding the recombinant lectin protein can be synthesised using chemical or recombinant means and expressed in a suitable host to obtain the recombinant proteins. Suitable host cells include prokaryotic cells and both lower eukaryotic cells as well as higher eukaryotic cells. Introduction of the recombinant molecule into the host cells can be achieved using methods known in the art. In an exemplary embodiment of the present invention the suitable host is a microbial cell. In a preferred embodiment, the microbial cell is selected from the group consisting of, but not limited to, a yeast cell, Escherichia coli, an insect cell line or a mammalian cell line. Further the recombinant proteins can be obtained by isolation, as an expression product from a recombinant host. In one embodiment, the recombinant proteins of the present invention, are purified by conventional techniques, typically chromatographic methods. Exemplarily the recombinant lectin protein of the present invention can be prepared by the processes disclosed in applicant's previous application WO/2020/074977.

In another embodiment of the present invention, the molecular mass of the recombinant lectins, determined by SDS-PAGE and Mass spectrometry, is approximate 16,000 Daltons.

According to an embodiment, the invention provides recombinant lectin for the treatment or prevention of neurodegenerative disease, wherein the treatment encompasses to reduce or eliminate or to diminish or to alleviate the signs and symptoms of the neurodegenerative disease and prevention comprises suppression, control or delay in the development or onset of neurodegenerative disease or symptoms related to disease.

According to some embodiments the neurodegenerative disease comprises the disease or disorder caused due to the degeneration of neurons or neuronal cells in and around brain or central nervous system.

The neurodegenerative disease may be caused due to enhanced or reduced levels of biomarkers such as ICAM-1/CD54, dopamine, serotonin, S100b, Park7/DJ-1, Calbindin D, B-NGF, RAGE, MPO, Tau, GDNF, α-synuclein, amyloid beta, Acetyline Cholinesterase, Periostin, Angiostatin converting enzyme (ACE), Thrombosporin-1, Plasma amyloid beta, VE-cadherin, LGALS3BP (Lectin galactoside binding soluble 3 binding protein), TNF-α (Tumor necrosis factor-Alfa). The increase or decrease in the normal levels of these biomarkers may indicate the onset or presence of neurodegenerative disease in the body under examination.

For example, the S100b is a calcium binding protein that plays vital role in pathogenesis of Parkinson's disease. The normal level of S100b in a normal human being without Parkinson's disease would be 10 μg/mL to 150 μg/mL, whereas a human being suffering from Parkinson's disease would show the amounts from 200 μg/mL and above. Thus, a human subject suffering from Parkinson's disease would have enhanced levels of S100b.

According to an aspect of the invention, recombinant lectin protein is capable of lowering the levels of S100b.

Similarly, according to an aspect, the recombinant lectin protein of present invention is capable of modulating the levels of biomarkers according to the requirements of the cells of the body, and thus treating or preventing the progression of the disease.

In an aspect of the invention, the markers listed above might be responsible for cause of one or more than one neurodegenerative diseases. The recombinant lectin protein of the present invention is capable of modulating the levels of these biomarkers and therefore will be effective in the controlling of progression or onset of one of more disease or diseases.

In one embodiment of the present invention, the recombinant lectin having SEQ ID No: 1 aids in normalizing the level of biomarker dopamine and serotonin in the neurotoxin damaged cells thereby restoring the cognitive health of the brain. As dopamine and serotonin hormone are capable of transmitting signals to nerve cells and responsible for maintaining sleep cycle, muscle contraction, mood functions, motor, autonomic functions. The cognitive health refers to the health of the overall brain, tissues and blood supply as well as its ability to function appropriately under various conditions. Good cognitive health is vital for the brain to perform all mental processes collectively known as cognition including, but not limited to, learning, intuition, judgment, language, attention, alertness, focus and memory (both long and short-term). Poor cognitive health due to aging, diseases and/or other cognitive detriments reduce the brain's ability to function appropriately resulting in significant declines in cognitive function and performance. Some of the cognitive health related disorder are Panic disorder, obsessive compulsive disorder (OCD), attention deficit hyperactivity disorder (ADHD), season effective disorder (SAD), sleep disorder, memory loss or disruption, stress, and depressed mood. During the pathogenesis of the Parkinson's disease the normal synthesis of Serotonin and Dopamine is highly affected and leads to Cognitive health disorder.

In some embodiment of the present invention, there is provided a method for the treatment or prevention of neurodegenerative disease, comprising administration of an effective amount of recombinant lectin protein derived from sclerotium rolfsii lectin to the subject.

The subject may be a mammalian subject. In some embodiments, the subject is human. In particular, the subject may be a human subject suffering from or seeking prevention from neurodegenerative disease.

In an embomidment the method of treatment or prevention of neurodegenerative disease comprises administration of therapeutically effictive amount of recombinant lectin protein derived from sclerotium rolfsii lectin, wherein the therapeutically effictive amount of lectin may be in the dose range from 0.01 mg/kg to 1000 mg/kg of the weight of the subject. In some embodiment the dose range may be from 0.1 mg/kg to 500 mg/kg or from 0.5 mg/kg to 100 mg/kg or from 1 mg/kg to 50 mg/kg. It will be within the capabilities of the skilled person to determine an amount of lectin to be administered according to the nature of the condition being treated and the subject.

In some embodiments, the recombinant lectin protein of the present invention may be administered as such or in the form of a pharmaceutical composition.

Thus, the present invention also provides a pharmaceutical composition for the treatment or prevention of neurodegenerative diseases comprising a recombinant lectin protein derived from sclerotium rolfsii lectin and pharmaceutically acceptable excipients. Exemplary excipients include sterilised water, physiological saline, and/or pharmaceutically acceptable buffer.

The composition may further comprise protein stabilizing agents, polymers, solubilizers, cryoprotectants, lyoprotectants, bulking agent/s diluents or mixture thereof. The composition may comprise the excipients listed in applicants co-pending Indian application No. 201921027358, which is incorporated in this application in its entirety by a way of reference.

Administration of the lectin protein or composition may be by any suitable route as understood by the skilled person, including but not limited to, injection (including intravenous (bolus or infusion), intra-arterial, intraperitoneal, subcutaneous (bolus or infusion), intraventricular, intramuscular, or subarachnoidal), oral ingestion (e.g. of a tablet, gel, lozenge or liquid), inhalation, topical, via a mucosa (such as the oral, nasal or rectal mucosa), by delivery in the form of a spray, tablet, transdermal patch, subcutaneous implant or in the form of a suppository.

In some embodiments, a lectin (such as a lectin having the amino acid sequence of SEQ ID NO: 1, 2, 3, or 4) or a pharmaceutical composition as described herein is administered to the subject enterally, parenterally or topically. The lectin or pharmaceutical composition may be administered as a dosage form which is solid (such as tablet or capsule), a lyophilized powder, a liquid (such as solution or suspension), a semi-solid or any other form as known to the person skilled in the art. The lectin or the pharmaceutical composition may be administered to the subject by injecting a solution or suspension intravenously, intramuscularly, intraperitoneally, subcutaneously, or intradermally, by depot injection, or it may be administered intrathecally, transdermally, sublingually or by oral, topical or inhalation methods.

As understood by the skilled person the suitable form of the composition may be determined by the route of administration of the composition. Therefore the suitable form of the composition may include but is not limited to, injection for intravenous (bolus or infusion), intra-arterial, intraperitoneal, subcutaneous (bolus or infusion), intraventricular, intramuscular, or subarachnoidal route; tablet, capsule, gel, lozenge or liquid for oral ingestion; a solution, suspension or aerosol as sprays for inhalation; gel, spray or cream for topical application; transmucosal composition for administration via oral, nasal or rectal mucosa; by delivery in the form of a transdermal patch, subcutaneous implant, or in the form of a suppository. The lectin protein may also be formulated in rectal compositions such as suppositories or retention enemas. For buccal administration, the compositions may take the form of tablets or lozenges. The composition may be a vesicular drug delivery system such as, but not limited to, bilosomes, liposomes, niosomes, transferosome, ethosomes, sphingosomes, pharmacosomes, multilamellar vesicles, microspheres and the like.

The composition of the present invention may be formulated as per understanding and the knowledge of the skilled person.

In an embodiment, the present invention provides the use of recombinant lectin protein derived from sclerotium rolfsii lectin for the treatment or prevention of neurodegenerative disease.

According to the embodiment the use may be of the recombinant lectin protein as such or in the form of composition comprising lectin protein and a pharmaceutically acceptable excipient.

In an embodiment, the present invention provides a method of inducing neuronal outgrowth by administering an effective amount of recombinant lectin protein derived from sclerotium rolfsii lectin.

According to the present invention ‘inducing of neuronal outgrowth’ indicates induction of growth of neurites, wherein the neurites are the projections from the cell body of a neuron. The lectin proteins of the present invention are capable of growing neurites in neurons, when administered to the subject in need thereof.

In one specific embodiment of the present invention the recombinant lectin protein may be selected from the lectin having sequence of SEQ ID NO: 1, SEQ ID NO: 2, SEQ ID NO: 3 or SEQ ID NO: 4.

In an embodiment, the neurodegenerative diseases treated or prevented according to the present invention are those listed herein above.

In particular embodiment the neurodegenerative diseases may be but not limited to Dementia such as Alzheimer's disease, Frontotemporal dementia (picks disease), Lewy body dementia, neurofibrillary tangle dementia, and symptoms related to dementia, Creutzfeld-Jacob disease (which has similar clinical manifestation as that of Alzheimer), Hippocampal sclerosis, Schilder's disease; Parkinson's disease, Parkinson like disease such as progressive supranuclear palsy (PSP), multiple system atrophy (MSA), and corticobasal degeneration (CBD); Ataxia, cognitive disorder, motor neuron disease like Amyotrophic lateral sclerosis (ALS), primary lateral sclerosis (PLS), progressive bulbar palsy (PBP) a variant of ALS, Pseudo bulbar palsy and Hereditary spastic paraplegia; Aneurysm, Epilepsy, and Huntington's disease, Pantothenate kinase-associated neurodegeneration (PKAN) Stroke, Batten Disease, Gerstmann-Straussler-Scheinker syndrome, CADASIL (Cerebral autosomal dominant arteriopathy with subcortical infarcts and leukoencephalopathy), Cerebellar Hypoplasia Cerebral Arteriosclerosis, Cerebral Hypoxia, Chorea, Chronic Inflammatory Demyelinating Polyneuropathy CIDP, Colpocephaly, globular glial tauopathies, primary age-related tauopathy, chronic traumatic encephalopathy (CTE), aging-related tau astrogliopathy, Leigh syndrome, Lewy body disease like diffused Lewy body disease (DLBD), genetic disease causing neuronal condition/loss such as Spinal muscular atrophy (SMA) a trinucleotide repeat genetic disorder, congenital sma with arthrogryposis a rare form of SMA, Spinal-Bulbar Muscular Atrophy (SBMA), polyglutamine (polyQ)-repeat diseases, Primary progressive aphasia (PPA), Alexander disease, Apler's disease, Krabbe's disease, Sandhoff disease, Hallervorden-Spatz syndrome and Kugelberg-Welander disease.

In a very particular embodiment, the neurodegenerative disease may be selected from Alzheimer's disease, Parkinson's disease, dementia, cognitive disorder and symptoms related to dementia, Amyotrophic lateral sclerosis (ALS) Lewy body disease, Spinal muscular atrophy and Huntington's disease.

In another very specific embodiment, the neurodegenerative disease may be selected from Alzheimer's disease, Parkinson's disease, dementia, cognitive disorder and symptoms related to dementia.

The present invention related to method of treatment or prevention of neurodegenerative disease using recombinant lectin protein having sequence of SEQ ID NO: 4 or its homologues. In vitro studies of recombinant lectin having sequence of SEQ ID NO: 1 demonstrated significant positive effect on the neuronal cell lines in presence of neurotoxin. The recombinant lectin protected neuronal cells from neurotoxin. Similarly, when effect of recombinant lectin having sequence of SEQ ID NO: 1 on biomarkers related to neurodegenerative disease was studied, it indicated the lectin modulated biomarker to the normal range from the abnormal range in the diseased state.

In particular, the In vitro studies on several cell lines for Parkinson's and Alzheimer's or Dementia was performed.

The cytoprotective effect of the recombinant lectin protein having SEQ ID NO: 1 was elucidated by determining cell viability in human neuronal cell line SH-SY5Y in presence of neurotoxin 1-methyl-4-phenylpyridinium Iodide (MPP+Iodide). The cell viability and restoration of cell viability against the neurotoxin induced cytotoxicity was determined. SEQ ID NO: 1 with concentration from 0.001 μg/mL to 50 μg/mL demonstrated 40% to 86% of cytoprotection as compared to positive control Deprenyl with concentration 1 μM to 100 M showed around 41% to 76% of cytoprotection.

The cytoprotective effect was studied by initially treating the SH-SY5Y cell lines with non-cytotoxic concentrations of recombinant lectin protein having SEQ ID NO: 1 and then the cell lines were exposed to neurotoxin MPP+ iodide. Deprenyl was used as positive control. Increase in cell viability between 78.8%-94.9% of recombinant lectin having SEQ ID NO: 1 treated Parkinson induced SH-SY5Y cells was observed as compared to positive control Deprenyl, which showed cell viability ranges between 79.3%-91.3%.

Effect of SEQ ID NO: 1 was evaluated to study the anti-apoptotic effect on human neuronal (SH-SY5Y) cell line. 1-methyl-4-phenylpyridinium Iodide (MPP+Iodide) was used as a neurotoxin to induce Parkinson's disease in SH-SY5Y cell lines. The in vitro anti-apoptotic effect of recombinant lectin having SEQ ID NO:1 was determined using three different studies, Annexin V staining technique, Sub G0/G1 by PI stain.

The anti-apoptotic effect of recombinant lectin protein of SEQ ID NO: 1 is assessed via restoration of mitochondrial membrane potential in SH-SY5Y cells against MPP+ iodide induced damage. The recombinant lectin protein having SEQ ID NO: 1 exhibited an anti-apoptotic effect with an increase in the mitochondrial membrane potential by 22.7%-115.9% at a concentration range from 1 μg/mL to 50 μg/mL.

The positive control showed 40% to 95% of increase in the mitochondrial membrane potential at concentration from 1 μM to 100 μM.

Further reduction of apoptotic cells using Annexin positive staining against the neurotoxin, MPP+ iodide was determined. Recombinant lectin protein exhibited decrease of apoptotic cells by population from 23.5%-70.4% at a concentration from 0.001 μg/mL to 1 μg/mL as compared to cells that were not treated with lectin and were exposed to neurotoxin. The positive control Deprenyl showed decrease of population from 27.2% to 38.3% at concentration from 10 μM to 100 μM.

Anti-apoptotic effect of SEQ ID NO: 1 was further confirmed by evaluating the Sub (G0/G1) cell population on human neuronal (SH-SY5Y) cell line against the neurotoxin, MPP+ iodide. The study exhibited decrease in apoptotic in Sub (G0/G1) cell population from 19% to 35% at a concentration ranging from 0.001 μg/mL to 1 μg/mL as compared to cells that were not treated with lectin and were exposed to neurotoxin. The positive control Deprenyl showed decrease of population from 13% to 42% at concentration from 10 μM to 100 μM. The neuroprotective efficacy of the recombinant lectin having SEQ ID NO: 1 is further validated by assessing the cognitive health with the aid of Neurite Outgrowth Assay using neuronal cells PC12 (Rat Pheochromacytoma cells). The cells were treated with Nerve growth factor (NGF), recombinant lectin having SEQ ID NO: 1 and MPP+ iodide and tested for neuroprotective effect. The study demonstrated that the recombinant lectin having SEQ ID NO: 1 possess a potent neuroprotective property, as it aids in restoration of neurite growth by 7.3%-78.2% in MPP+ iodide induced damage in cells at concentration from 0.0001 μg/mL to 5 μg/mL. On the contrary the cells that were treated with only NGF and MPP+ iodide demonstrated 0% of protection in the formation of neurites.

It is evident from the above studies that the recombinant lectin protein having SEQ ID NO: 1 possess a cytoprotective effect which prevents the neural cells from neurotoxins and decreases apoptosis in the cells considerably. Thus, recombinant lectin protein of the present invention prevents the cells and therefore the body from onset of the disease by aiding in viability of the neural cells when exposed to neurotoxin.

Further studies were conducted to evaluate the effect of recombinant lectin protein having SEQ ID NO: 1 on the neurite growth. PC12 cells were treated with recombinant lectin having SEQ ID NO: 1 at concentrations ranging from 0.001 μg/mL to 5 μg/mL. The lectin exhibited increase in number of neurites from 19% to 55% as compared to the untreated cells. The recombinant lectin of present invention showed considerable neurite outgrowth and can be used for the said purpose for the subject in need of such treatment.

The mechanism of action of recombinant lectin having SEQ ID NO: 1 in Parkinson's disease was determined by multiplex analysis and ELISA and expression levels of biomarkers were evaluated. The test samples are prepared by treating the human neuronal (SH-SY5Y) cells with varying concentration of recombinant lectin protein having SEQ ID NO: 1 from 0.001 μg/mL to 1 μg/mL for 24 h and further treated with neurotoxin MPP+Iodide in-order to induce neuronal damage.

The study reveals that recombinant Lectin having SEQ ID NO: 1 restores the Parkinson's affected neuronal cells by activating neurotransmitter, neuroprotective signalling cascade proteins such as dopamine which increased from 22.9%-82.5%; Serotonin which increased from 5.1% to 23.5% and Calbindin D, the levels of which increased from 1.7%-9.7% as compared to the cells treated with MPP+ iodide. On the brighter side, the recombinant lectin having SEQ ID NO: 1 also possesses a remarkable inhibitory effect against inflammatory cell adhesion protein such as ICAM-1/CD54 levels of which decreased from 6.2%-41.3% and calcium binding protein S100b which decreased from 6.5%-11.4% as compared to the cells treated with MPP+ iodide. However, there was no significant modulation observed in the case of α-Synuclein.

The mechanism of action of recombinant lectin having SEQ ID NO: 1 in Alzheimer's disease was elucidated by multiplex analysis. The biomarkers assessed to delineate mechanism of action are b-NGF, RAGE, MPO, Tau. The mechanism of action of Alzheimer's was determined by treating the human neuronal (SH-SY5Y) cells with concentration of recombinant lectin protein having SEQ ID NO: 1 from 0.01 μg/mL to 25 μg/mL for 24 h and further treated with neurotoxin Scopolamine in order to induce neuronal damage.

The effect of recombinant lectin protein having SEQ ID NO: 1 supressing the Alzheimer's disease was assessed by the activation and inhibition of certain biomarkers such as b-NGF a neuronal growth factor biomarker whose levels increased from 41% to 89% as compared to the Scopolamine treated cells; MPO (metalloperoxidase) and RAGE (Receptor for advanced glycation end products) were inhibited by SEQ ID NO: 1, showing decrease in their levels from 46% to 64% and 3%-19%, respectively. The inhibition of MPO (metalloperoxidase) and RAGE (Receptor for advanced glycation end products) demonstrates the significance of therapeutic effect of recombinant lectin against Alzheimer's disease. RAGE plays a crucial role in prognosis of Alzheimer's disease, since it is a multiligand surface molecule of immunoglobulin superfamily serves as receptor for amyloid-beta protein. Increase expression of RAGE due to genetic aberration results in neural inflammation with generation of reactive oxygen species leading to oxidative stress. MPO is an immunoregulatory protein plays a vital role in induction of cytokines and it majorly catalyses the conversion of hydrogen peroxide to hypochlorous acid in presence of chloride, which leads to formation of oxidation adducts by reacting with biological species. Abnormality in MPO gene results in overexpression of MPO in frontal cortex region of brain leading to regeneration.

In vitro studies suggested the safety and therapeutic effectiveness of recombinant lectin protein against neurodegenerative diseases. The therapeutic effect of recombinant lectin protein was further validated by determining its In-vivo Anti-dementia activity using Scopolamine induced dementia in Swiss albino mice, by measuring the transfer latency time in passive avoidance test, changes in levels of brain biomarkers and histopathology of hippocampus region of the brain.

The cognitive-enhancing activity of recombinant lectin protein having SEQ ID NO 1 against scopolamine-induced memory impairments in mice was determined using the passive avoidance test. The animals injected with scopolamine Group G2 showed significant decrease (p<0.001) in transfer latency time, (i.e. 56.93±5.12 Sec.) when compared with the normal control Group G1, (i.e. 135.70±5.11 Sec.) indicating the short-term memory deficit in mice. The animals treated with Donepezil (G3, 2.5 mg/kg) showed significant increase (p<0.001) in transfer latency time, (i.e. 110.61±7.02 Sec.) when compared with the G2 indicates the scopolamine-induced short-term memory deficit was reversed to normal by the choline esterase inhibitor donepezil. The animals treated with recombinant lectin protein having SEQ ID NO 1 at three dose levels, Group G4 (0.5 mg/kg) showed significant increase (p<0.05) (85.26±6.52). G5 (0.25 mg/kg) showed increase of 80.58±9.08 Sec. and G6 (0.125 mg/kg) showed improvement 75.81±7.75 Sec. in memory when compared to G2.

Percentage increase in transfer latency time was 48.5% for an animals treated with donepezil as compared with untreated. The animals treated with the recombinant lectin protein having SEQ ID NO 1 G4, G5 and G6 with different doses showed increase in transfer latency time 33.2%, 29.4% and 24.9% respectively when compared with G2.

Further the Brain biomarker such as Brain NGF (Nerve Growth Factor), TNF alpha and Acetylcholinesterase (AChE) levels were estimated by ELISA.

NGF (nerve growth factor) plays a pivotal role in neuronal plasticity and neurogenesis via the inhibition of cAMP response element binding protein (CREB, where CAMP is cyclic adenosine monophosphate) phosphorylation6. Impaired CREB phosphorylation is a known pathological factor of neurodegenerative disorders, triggering neuronal loss in the hippocampus and cortex via a pro-apoptotic process. Inhibition of CREB impairs behavioural performance on various memory tests. In contrast, overexpression of CREB promotes neuronal survival and ameliorates cognitive impairments via the cholinergic system. The neurotoxin, scopolamine treatment significantly (p<0.001) reduces NGF expression in the brain when compared with levels observed in the normal control group. However, the recombinant lectin protein having SEQ ID NO: 1 treated groups of varying dose of 0.5 mg/kg and 0.25 mg/kg showed significant (p<0.001) increased level of NGF (71.6 pg/mL and 62 μg/mL, respectively) when compared with scopolamine treated dementia group (34.5 pg/mL). Donepenzil (Positive control) treated group showed increase of 57 μg/mL. The study results indicate that recombinant lectin protein having SEQ ID NO: 1 protect or ameliorate learning and memory impairments by activating neurotrophic factors and preventing neuronal apoptosis. Further AChE activity of the brain in study group was evaluated. The study reveals that Scopolamine group significantly (p<0.001) increased AChE activity in the brain suggesting that the observed cognitive impairments were induced by cholinergic dysfunction. However, pretreatment of recombinant lectin protein having SEQ ID NO: 1, 0.5 mg/kg significantly (p<0.001) attenuated these scopolamine-induced impairments. AChE, is well-known enzyme which plays a pivotal role in learning and memory. Choline acetyltranferase (ChAT) is a critical cholinergic marker participating in Acetylcholine, (Ach) synthesis. Maintenance of Ach is essential for normal function, whereas inordinate AChE activity results in disruptions in Ach levels in the brain. Acetylcholine signaling eventually derives the phosphorylation of the cAMP (cyclic adenosine monophosphate) response element binding protein (CREB), which then translocates into the nucleus to regulate the transcription of target genes. It is well known that CREB plays a crucial role in neuronal growth, proliferation, differentiation and survival. Numerous studies have also emphasized the interrelationship between the transcriptional activity of CREB and hippocampus-dependent memory formation.

The study group of animals that were treated with neurotoxin scopolamine showed AChE level of 6.74 mU/mL, whereas the positive control, that is study group treated with scopolamine and Donepezil showed AChE level of 4.39 mU/mL. The study group treated with both scopolamine and SEQ ID NO: 1, showed AChE level of 4.47 mU/mL at 0.5 mg/kg and 6.27 mU/mL at 0.25 0.5 mg/kg and 6.59 mU/mL at 0.125 mg/kg. In the untreated group the level of AChE was 4.19 mU/mL

In a further study, Scopolamine-induced dementia increased level of brain TNF-α indicating mild to moderate neuroinflammation. Scopolamine group showed significant (p<0.01) increase in brain TNF-α level (144 pg/mL); meanwhile Recombinant Lectin having SEQ ID NO: 1 in dose levels 0.5 mg/kg (121.7 pg/mL), 0.25 mg/kg (67.65 pg/mL) and 0.125 mg/kg (79.53 pg/mL) significantly decreased brain content of TNF-α suggesting the anti-inflammatory activity. The normal group without scopolamine treatment has 61.02 pg/mL and the positive control group showed treated with donepezil and scopolamine showed 156 μg/mL level of TNF-α

Detailed histological investigation was performed on H&E (Hematoxylin and Eosin) stained brain tissues in Cerebral Cortex and Hippocampus (CA1, CA3 and DG) regions. The results showed that no significant changes were observed in all the treatment groups as well as scopolamine injected group in cerebral cortex and hippocampus region except the CA3 region. The significant (p<0.01) damage was found in the CA3 region of hippocampus in group G2 i.e. scopolamine disease control group which showed mean score value of 2.0 when compared with normal control group (0). Non-significant decrease was observed in G3 that is Donepezil treated group, 1.33 when compared with G2. Moreover, the group G5-treated with recombinant lectin protein having SEQ ID NO: 1 of 0.25 mg/kg showed significant (p<0.05) decrease value i.e. 0.60. and the groups G4 (0.5 mg/kg of SEQ ID NO: 1) and G6 (0.125 mg/kg of SEQ ID NO:1) showed decrease in mean score value i.e. 0.67 and 1.00, respectively. It is well known to the skilled person that the hippocampus is a pivotal region of the brain for learning and memory. Adult hippocampal neurogenesis plays an important role in memory formation; therefore, impaired neurogenesis and neuronal integration are regarded as pathological features of neurodegenerative disorders. Thus, recombinant lectin protein having SEQ ID NO: 1 treatment markedly reversed the scopolamine induced inhibition of neurogenesis in the hippocampal structure in CA3. This neurogenesis is known to depend on the activities of both neurotrophins and their receptors.

According to an embodiment of the present invention, the recombinant lectin protein derived from sclerotium rolfsii lectin having SEQ ID NO: 1 prevents the neuronal cells from apoptosis due to excitotoxicity and restores the function of the neurons by a neural growth in disease induced animal models. Excitotoxicity can occur from toxic substances of endogenous or exogenous origin which is the key mechanism in neurodegerative disease such as Alzheimer, Parkinson, Huntington's disease, Amyotrophic lateral sclerosis (ALS), dementia etc. Excitotoxicity is the over activation of the neurotransmitters that primarily causes severe damages to nerve cells by affecting the mitochondrial functions which in turn results in oxidative stress. The excess influx of calcium ions may also be one of the mechanisms involved in neuronal loss. As a result of this, the neuronal cells lose their functions and are degenerated by apoptosis. Therefore the recombinant lectin protein derived from sclerotium rolfsii lectin having SEQ ID NO: 1 has therapeutic potency for the treatment or prevention of neurodegerative disease such as Alzheimer, Parkinson, Huntington's disease, Amyotrophic lateral sclerosis (ALS), dementia, where it prevents the neuronal cells from apoptosis by restoring the mitochondrial membrane potentials; regulating the expression levels of certain biomarkers levels which plays key role in functions of the neurons and also aiding in neuronal growth.

In further aspect to the embodiment of the present invention the lectin protein derived from sclerotium rolfsii lectin exhibited therapeutic efficacy and was effective in prevention and treatment of neurodegenerative disease such as Alzheimer's, Parkinson's and Dementia or symptoms related to dementia. The lectin further exhibited effective in neurite outgrowth and restoration of cognitive functions in the disease induced models.

### EXAMPLES

The following examples are given to demonstrate the best mode of performance of the invention. Examples do not limit the invention in any manner.

SH-SY5Y-Cells are derived from sub cloned cell-line of SK-N-SH human neuroblastoma cells. It serves as a model for neurodegenerative disorder, as the cell can be modified to various types of functional neurons by adding specific compounds. Hence this property of the SH-SY5Y cell lines made them a suitable model for experimental neurological studies, including analysis of neuronal differentiation, metabolism, and function related to neurodegenerative processes, neurotoxicity, and neuroprotection. The Human Neuronal cell line SH-SY5Y was procured from National Centre for Cell Science (NCCS), Pune.

The SH-SY5Y cell line was maintained in DMEM: Ham's F12 (1:1)+10% FBS growth medium under growth condition 5% CO₂, 37° C. and 95% humidity. The cell line was sub-cultured by splitting the cell suspension into fresh flasks and supplementing with fresh culture medium.

PC-12 Cells The adrenal phaeochromocytoma (PC12) cell line was originally isolated from the adrenal medulla of the rat. The ability of PC12 cells to synthesise and store dopamine and resemble sympathetic ganglion neurons upon differentiation with nerve growth factor (NGF) made them suitable model for the Parkinson's disease. The Rat Pheochromacytoma cells PC12 was procured from American Type Culture Collection (ATCC) USA.

The PC12 cell line was maintained in Ham's F12+10% FBS growth medium under growth condition 5% CO₂, 37° C. and 95% humidity. The cell line was sub-cultured by trypsinization and splitting the cell suspension into fresh flasks and supplementing with fresh culture medium.

Aqueous solution of recombinant lectin protein having the sequence having SEQ ID NO: 1 was provided. The stock solution was diluted in Serum Free Medium (SFM) to achieve final concentrations.

### Example 1: Evaluation of Cytoprotective Effect of SEQ ID NO: 1 in Neuronal Cells for Beneficial Effect in Parkinson's Disease

The cytoprotective effect of SEQ ID NO: 1 in SH-SY5Y neuronal cells was conducted using following assay.

The Human Neuronal SH-SY5Y Cells were counted using hemocytometer and plated in 96 well plate at a density of 25,000 cells/well and incubated at 37° C. for 24 h. After incubation, cells were treated with recombinant lectin protein having the sequence of SEQ ID NO: 1 at concentrations ranging from 0.001 μg/ml 50 μg/ml for 24 h. After 24 h of treatment, cells were exposed to neurotoxin (MPP+ iodide, 1 mM) for 24 h. Cells treated with MPP+ iodide alone were included as negative control. Untreated cells were included as control. Cells treated with Deprenyl (1 μM-100 μM) served as positive control. After treatment, the cytoprotective effect of SEQ ID NO: 1 on cell viability was assessed by 3-(4,5-dimethythiazol-2-yl)-2,5-diphenyl tetrazolium bromide (MTT) assay: The plate was taken out and 20 μl of 5 mg/ml of MTT 3-(4,5-dimethythiazol-2-yl)-2,5-diphenyl tetrazolium bromide solution was added to all the wells. The SH-SY5Y cells were incubated for 3 h at 37° C. The supernatant was aspirated and 150 μl of DMSO was added to each well to dissolve formazan crystals. The absorbance of each well was read at 540 nm using Synergy HT micro plate reader. The protective effect of the SEQ ID NO: 1 on survival of SH-SY5Y cells against MPP+ iodide induced damage was determined as: The viability of cells was determined as:

$$\% Cell viability = ( 100 - \% Cytotoxicity ) ;$$

Where, the percentage cytotoxicity corresponding to each treatment was calculated as follows:

$$\% Cytotoxicity = [ ( R - X ) / R ] * 100$$

X=Absorbance of cells treated with MPP+ iodide/recombinant lectin having SEQ ID NO: 1+MPP+Iodide
R=Absorbance of Control cells (Untreated)
Percentage protection was calculated as:

[(Absorbance of SEQ ID NO:1+MPP+Iodide)−(Absorbance of MPP+Iodide)/(Absorbance of Untreated)−(Absorbance of MPP+Iodide alone)]*100

TABLE 1
Cytoprotective effect of Recombinant lectin having SEQ ID NO: 1 in
neuronal cells (SH-SY5Y) against neurotoxin (MPP+) induced damage

| | | | | Percentage | |
|---|---|---|---|---|---|
| | | | Percentage | Viability | Percentage |
| Neurotoxin | Sample | Concentration | cytotoxicity | (w.r.t control) | Protection |
| Untreated | | | 0 | 100 | |
| MPP + Iodide(1 mM) | | | 35.4 | 64.6 | 0 |
| MPP + | Deprenyl | 1 | 20.7 | 79.3 | 41.5 |
| Iodide | (μM) | 10 | 18.1 | 81.9 | 49 |
| (1 mM) | | 25 | 8.7 | 91.3 | 75.6 |
| | | 50 | 9 | 91 | 74.7 |
| | | 100 | 11.5 | 88.5 | 67.5 |
| | SEQ ID | 0.001 | 14.9 | 85.1 | 58.6 |
| | NO: 1 | 0.01 | 13.7 | 86.3 | 61.8 |
| | (μg/mL) | 0.05 | 11.2 | 88.8 | 68.8 |
| | | 0.1 | 17 | 83 | 52.6 |
| | | 1 | 21.2 | 78.8 | 40.2 |
| | | 5 | 17.9 | 82.1 | 49.6 |
| | | 10 | 5.1 | 94.9 | 85.6 |
| | | 25 | 7.6 | 92.4 | 78.6 |
| | | 50 | 13.4 | 86.6 | 62.1 |

### Example 2: Evaluation of Anti-Apoptotic Effect of Recombinant Lectin Having SEQ ID NO: 1 in Neuronal Cells for Beneficial Effect in Parkinson's Disease

The anti-apoptotic effect of recombinant lectin having SEQ ID NO: 1 in neuronal cells for beneficial effect in Parkinson's disease was determined using following assay.

### Example 2a: Determination of Effect of Recombinant Lectin Having SEQ ID NO: 1 on Mitochondrial Membrane Potential

Cells were counted using hemocytometer and plated in black well, 96-well plate at a density of 25,000 cells/well of the complete growth medium. The plated cells were incubated overnight in 5% CO2 incubator at 37° C. After 24 h, cells were treated with concentrations of recombinant lectin protein having SEQ ID NO: 1 ranging from 1 μg/ml-50 μg/ml for 24 h. After 24 h of treatment, cells were exposed to damage (MPP+ iodide 1 mM) for 24 h. Cells treated with MPP+ iodide alone were included as negative control. Untreated cells were included as control. Cells treated with Deprenyl served as positive control. After MPP+ iodide exposure, the protective effect of recombinant lectin protein having SEQ ID NO: 1 on mitochondrial membrane potential was assessed by JC-1 assay as follows; after incubation, the supernatants were discarded and 100 μl of JC1-dye solution (prepared by diluting 1 mM DMSO stock in to 10 μM in 1×PBS) was added to each well. The cells were then incubated with the dye in CO2 incubator at 37° C. for 15 min. After 15 min of incubation, the supernatant was removed, and the cells were washed twice with 1×PBS (phosphate buffer saline). 100 μl of 1×PBS was finally added to each well. Red fluorescence (excitation 550 nm, emission 600 nm) and green fluorescence (excitation 485 nm, emission 535 nm) were measured using Biotek Synergy HT plate reader. The mitochondrial membrane potential (Δψm) was calculated as the ratio of intensity of red fluorescence to intensity of green fluorescence described as follows:
Δψm=Intensity of red fluorescence/Intensity of green fluorescence
The percentage increase/restoration in Mitochondrial Membrane Potential against MPP+ iodide damage was calculated as:

$$\% Increase = [ ( R - X ) / R ] * 100$$

Where X=Δψm corresponding to SEQ ID NO: 1+ MPP+ iodide treated cells
R=Δψm corresponding to control cells (MPP+ iodide damage alone)

TABLE 2
Anti-apoptotic effect of SEQ ID NO: 1 in neuronal
cells (SH-SY5Y) via restoration of mitochondrial
membrane potential against MPP + iodide damage

| | | | | % Increase in |
|---|---|---|---|---|
| | | | Mitochondrial | Mitochondrial |
| | | | membrane | potential (w.r.t. |
| Sample | | Concentration | potential | control) |
| Untreated | | | 3.1 | |
| MPP + Iodide (1 mM) | | | 1.8 | 0 |
| MPP + | Deprenyl | 1 | 2.9 | 58.2 |
| Iodide | (μM) | 10 | 3.1 | 67.9 |
| (1 mM) | | 25 | 3.6 | 95.3 |
| | | 50 | 3.3 | 79.6 |
| | | 100 | 2.6 | 39.9 |
| | SEQ ID | 1 | 2.3 | 22.7 |
| | NO: 1 | 5 | 3.4 | 86.6 |
| | (μg/mL) | 10 | 4 | 115.9 |
| | | 25 | 3.4 | 84.7 |
| | | 50 | 3.3 | 77.5 |

### Example 2b: Determination of Effect of Recombinant Lectin Protein Having SEQ ID NO: 1 on Annexin-V Staining

After incubation, cells were treated with recombinant lectin protein having SEQ ID NO: 1 at concentrations ranging from 0.001 μg/ml-1 μg/ml for 24 h. After 24 h of treatment, cells were exposed to damage (MPP+ iodide 1 mM) for 24 h. Cells treated with MPP+ iodide alone were included as negative control. Untreated cells were included as control. Cells treated with Deprenyl served as positive control. After treatment, cells were harvested by trypsinization and processed for Annexin V assay as follows: Cell were gently harvested into pre-labeled sterile centrifuge tubes and centrifuged at 300×g for 5-7 min. After 24 h of treatment, cells were exposed to damage (MPP+ iodide 1 mM) for 24 h. Cells treated with MPP+ iodide alone were included as Control. Untreated cells were included as negative control. Cells treated with Deprenyl served as positive control. After treatment, cells were harvested by trypsinization and processed for Annexin V assay as follows: Cell were gently harvested into pre-labelled sterile centrifuge tubes and centrifuged at 300×g for 5-7 min. Supernatant were discarded and the pellet was resuspended in 200 μl of fresh culture medium. 100 μl of cell suspension was transferred into pre-labeled sterile centrifuge tubes. 100 μl of Annexin-V reagent was added to each tube and incubated for 30 min at RT in dark. Cells stained for Annexin-V were then transferred into 96-well plates and acquired on flow cytometer (Guava technologies). Percentage of Annexin-V positive cells was determined.

Percent inhibition in apoptotic cells=[(% Annexin positive cells in MPP+iodide alone)−(% Annexin positive cells in Recombinant Lectin having SEQ ID NO:1+MPP+iodide)/% Annexin positive cells in MPP+iodide alone]*100

TABLE 3
Anti-apoptotic effect of SEQ ID NO: 1 in neuronal
cells (SH-SY5Y) via decrease in Annexin positive
cell population against MPP + iodide damage

| | | | | % Decrease in |
|---|---|---|---|---|
| | | | % Apoptotic | Apoptotic cells |
| | | | cells | (wrt MPP + |
| Sample | | Concentration | (Anenxin + ve) | iodide) |
| Untreated | | | 0.9 | |
| MPP + Iodide (1 mM) | | | 4.1 | 0 |
| MPP + | Deprenyl | 10 | 3 | 27.2 |
| Iodide | (PC) | 25 | 2.5 | 38.3 |
| (1 mM) | (μM) | 100 | 2.9 | 28.4 |
| | SEQ ID | 0.001 | 1.8 | 56.8 |
| | NO: 1 | 0.01 | 1.2 | 70.4 |
| | (μg/mL) | 0.05 | 2.6 | 35.8 |
| | | 0.1 | 3.1 | 23.5 |
| | | 1 | 2.3 | 43.2 |

### Example 2c: Anti-Apoptotic Effect of Recombinant Lectin Protein of SEQ ID NO: 1 in Neuronal Cells (SH-SY5Y) Via Decrease in Sub (G0/G1) Cell Population Against MPP+ Iodide Induced Damage

After incubation as in Example 2a and 2b, cells were harvested by trypsinization and processed for Cell cycle assay as follows: Cell cycle reagent contains PI stain, which stains DNA of cells in different phases of cell cycle; Sub (G0/G1), G1, S, G2 and M. Cells in Sub (G0/G1) phase correspond to apoptotic cells.

Cells were gently harvested into pre-labelled centrifuge tubes and centrifuged at 450 g for 5 min, RT (low brake). The supernatants were removed carefully and discarded. 1 ml of 1×PBS was added to the pellet and resuspended gently to make homogenous suspension. Cells were centrifuged at 450 g for 5 min, RT (low brake). The supernatant was carefully removed leaving behind approximately 100 μl of PBS. Cells were resuspended gently yet thoroughly in residual PBS. The cells were fixed by adding Ice-cold 70% ethanol (100 μl) added dropwise into cells in each tube while vortexing at low speed Cells were stored at 4° C. for 24 h prior to staining.

Ethanol fixed cells were centrifuged at 450 g for 5 min, RT (low brake) the supernatant was carefully removed (not to touch the pellet) and discarded. 1 ml of 1×PBS was added into pellet and resuspended gently. Cells were incubated for 1 minute at RT. Cells were centrifuged at 450 g for 5 min, RT (low brake). (washing step) The supernatant was removed carefully leaving behind approx. 20 μl-50 μl of PBS. 200 μl of Cell Cycle reagent was added into each tube. Cells were resuspended gently and mixed. Cells were incubated for 30 min, RT, Dark. The stained samples were transferred into 96-well plates and acquired on flow cytometer (Guava technologies). Percentage of cells in Sub (G0/G1) phase were determined.

Percent inhibition in apoptotic cells=[(% Sub(G0/G1)cells in MPP+iodide alone) −(% Sub(G0/G1)cells in SEQ ID NO:1+MPP+iodide)/% Sub(G0/G1)cells in MPP+iodide alone]*100

TABLE 4
Anti-apoptotic effect of SEQ ID NO: 1 in neuronal
cells (SH-SY5Y) via decrease in Sub (G0/G1) cell
population against MPP + iodide induced damage

| | | | % Apoptotic | % Decrease in |
|---|---|---|---|---|
| | | | cells | Apoptotic cells |
| Sample | | Concentration | (SubG0/G1) | (wrt MPP + iodide) |
| Untreated | | | 1.9 | |
| MPP + Iodide (2 mM) | | | 3.4 | 0 |
| MPP + | Deprenyl | 10 | 2.9 | 12.8 |
| Iodide+ | (PC) | 25 | 2.4 | 28.3 |
| | (μM) | 100 | 2 | 42 |
| | SEQ ID | 0.001 | 2.7 | 18.8 |
| | NO: 1 | 0.01 | 2.2 | 33.5 |
| | (μg/mL) | 0.05 | 2.4 | 29.9 |
| | | 0.1 | 2.4 | 27.7 |
| | | 1 | 2.2 | 35 |

### Example 3: Evaluation of Effect of SEQ ID NO: 1 on Cognitive Health in Parkinson's Disease by Neurite Outgrowth Assay In Vitro

The Cognitive Health in Parkinson's disease By Neurite Outgrowth was studied by following methods:

### Example 3a: Effect of SEQ ID NO: 1 on Neurite Formation in Neuronal Cells (Pc12) in Basal Model

Cells were counted using haemocytometer and plated in 24-well plates at the densities corresponding to 1×104 cells/well/500 μl of the growth medium. The cells were then incubated for 48 h in 5% CO₂ incubator at 37° C. Effect of Recombinant Lectin having SEQ ID NO: 1 on neurite formation was evaluated in both basal and MPP+ damage induced model.

For basal model the percent increase in neurite formation was determined as follows

$$\% Increase = [ ( No . of Neurites in Treated cells - No . of Neurites in untreated cells ) / No . of Neurites in untreated cells ] \times 100$$

TABLE 5
Effect of recombinant lectin protein of SEQ ID NO: 1 on
neurite formation in neuronal cells (pc12) in basal model

| | | Average | % Protection in the |
|---|---|---|---|
| | | No. of | formation of Neurites |
| Sample | Concentration | Neurites | (w.r.t. MPP+) |
| Untreated | | 18 | 0 |
| Recombinant | 0.001 | 27 | 54.7 |
| lectin SEQ | 0.01 | 26 | 47.2 |
| ID NO: | 0.1 | 21 | 18.9 |
| 1 μg/ml | 5 | 24 | 34 |

### Example 3b: Protective Effect of SEQ ID NO: 1 on Neurite Formation in Neuronal Cells (Pc12) Against Mpp+ Induced Damage

The percent protection in neurite formation against MPP+ induced damage was determined as follows:

$$\{ ( A - B ) / ( C - B ) \} * 100$$

Where,
A=No of neurites in Cells treated with NGF+SEQ ID NO: 1+MPP+
B=No of neurites in Cells treated with NGF+MPP+
C=No of Neurites Cells treated with NGF alone

TABLE 6
Protective effect of recombinant lectin protein
having SEQ ID NO: 1 on neurite formation in neuronal
cells (pc12) against mpp+ induced damage

| | | Average | % Protection in |
|---|---|---|---|
| | | No. of | formation of Neurites |
| Sample | Concentration | Neurites | (w.r.t. MPP+) |
| Untreated | | 4 | 0 |
| NGF (200 ng/ml) | | 38 | 100 |
| NGF (200 ng/ml) + MPP (100 μM) | | 20 | 0 |
| Recombinant | 0.001 | 21 | 7.3 |
| lectin SEQ | 0.01 | 28 | 43.6 |
| ID NO: | 0.1 | 34 | 76.4 |
| 1 μg/ml | 1 | 34 | 78.2 |
| | 5 | 30 | 58.2 |

### Example 4: Elucidation of Mechanism of Action of Recombinant Lectin Protein Having SEQ ID NO: 1 in Parkinson's and Alzheimer's by Multiplex Analysis Culture and Maintenance of Cell Line

### A. Estimation of Markers by Multiplex Analysis/ELISA

After overnight incubation as in the previous examples, cells were treated with recombinant lectin protein having SEQ ID NO: 1 at different concentrations ranging from 0.001 μg/ml-1 μg/ml for 24 h for Parkinson disease and 0.01 μg/ml-25 μg/ml for 24 h for Alzheimer's disease.
Parkinson Disease: After 24 h of pre-treatment with recombinant lectin protein having SEQ ID NO: 1, cells were exposed (MPP+) for another 24 h. Cells treated with MPP+Iodide were included as Control. The cells treated with Deprenyl were included as positive Control.
Alzheimer's Disease: After 24 h of pre-treatment with recombinant lectin protein having SEQ ID NO: 1, cells were exposed to scopolamine (4 mM) for another 24 h. cells treated with scopolamine were included as Control. The cells treated with Galantamine were included as positive Control.

The levels of markers were determined by multiplex analysis as: Cell culture supernatants were diluted (1:2) with Calibrator Diluent. 50 μl of standard or sample was added per well. 50 μl of the microparticle cocktail was added to each well of the microplate and covered with a foil plate sealer. The plate was incubated for 2 hours at room temperature on a horizontal orbital microplate shaker. The plate was washed using a magnetic device designed to accommodate a microplate. Washing was done by applying the magnet to the bottom of the microplate, allowing 1 minute before removing the liquid, filling each well with wash buffer (100 μl) and allowing 1 minute before removing the liquid again. 50 μl of diluted Biotin-Antibody Cocktail was added to each well. Covered the plate with a foil plate sealer and incubated for 1 hour at room temperature on the shaker. Repeated the wash step. 50 μl of diluted Streptavidin-PE was added to each well. The plate was securely covered with a foil plate sealer and incubated for 30 minutes at room temperature on the shaker. Repeated the wash step. The microparticles were resuspended by adding 100 μl of Wash Buffer to each well. The plate was incubated for 2 minutes on the shaker and read within 90 minutes using Magpix® multiplex machine. Levels of biomarkers for Parkinson's and Alzheimer's disease were estimated using Magpix® multiplex machine.

For Parkinson's the percent modulation in each sample was determined as follows:

[{Concentration of biomarkers(pg/ml) in SEQ ID NO:1+MPP+Iodide treated cells)−Concentration of biomarkers(pg/ml) in Control cells(MPP+Iodide alone treated)}/Concentration of biomarkers(pg/ml) in Control cells(MPP+Iodide alone treated)]*100

For Alzheimer's disease the percent modulation in each sample was determined as follows:

$$\% Change = [ ( Conc . of biomarker in Control cells - Conc . of biomarker in Control cells ) / Conc . of analyte in Control cells ] \times 100$$

### Results

TABLE 7
Effect of recombinant lectin protein having SEQ
ID NO: 1 on expression of biomarkers associated with
Alzheimer's disease in Neuronal cell line (SH-SY5Y)
% Change of Biomarkers markers (wrt Control)

| Sample | | Concentration | | b-NGF | RAGE | MPO |
|---|---|---|---|---|---|---|
| Scopolamine (4 mM) | | | | 0 | 0 | 0 |
| Scopolamine | Galantamine | 1 | μM | 9.1 | 0 | −64 |
| (4 mM) | (μM) | 10 | μM | 0 | 0 | −31.2 |
| | | 100 | μM | −9 | −38.7 | −45.9 |
| | SEQ ID | 0.01 | μg/mL | 0 | −19.3 | 1.7 |
| | NO: 1 | 0.1 | μg/mL | 0 | 0 | −64 |
| | (μg/mL) | 1 | μg/mL | −9 | −19.3 | −45.9 |
| | | 10 | μg/mL | 41.1 | 16.3 | 0 |
| | | 25 | μg/mL | 88.9 | −3 | 0 |

TABLE 8
Effect of recombinant lectin protein having SEQ ID NO: 1 on expression of
biomarkers associated with Parkinson disease in Neuronal cell line (SH-SY5Y)
% Change of Biomarkers markers (with respect to Control)

| | | | | | | | Park7/ | ICAM1/ |
|---|---|---|---|---|---|---|---|---|
| Sample | Concentration | | Calbindin D | Dopamine | Serotonin | S100b | DJ-1 | CD54 |
| MPP + Iodide (2 mM) | | | 0 | 0 | 0 | 0 | 0 | 0 |
| MPP + Iodide | 10 | μM | 5 | 21.2 | 9.1 | −6.7 | −6.8 | 12.9 |
| treated with | 50 | μM | 0 | 37.7 | 33 | −6.5 | −7.9 | −23.7 |
| Deprenyl | 100 | μM | 3.3 | −47.2 | 17.1 | −6.5 | −9.2 | −37 |
| MPP + Iodide | 0.001 | μg/mL | 9.7 | 50.6 | 15.4 | −11 | −2.5 | 0 |
| treated with | 0.01 | μg/mL | 8.2 | 82.5 | 5.1 | 9 | −3.6 | −6.2 |
| SEQ ID NO: 1 | 0.05 | μg/mL | 1.7 | 24.7 | 6.9 | −6.7 | −7.1 | −28.1 |
| (μg/mL) | 0.1 | μg/mL | 6.6 | 28.9 | 7.9 | −6.5 | −10.4 | −36.3 |
| | 1 | μg/mL | −1.6 | 22.9 | 23.5 | −11.4 | −12.6 | −41.3 |

### In-Vivo Studies:

### Example 5: Recombinant Lectin Protein Having SEQ ID NO: 1

Required amount of recombinant lectin protein having SEQ ID NO: 1 was diluted in sterile Tris Buffered Saline (TBS) to achieve desired final concentration, i.e 0.1 mg/mL, 0.05 mg/mL, 0.025 mg/mL, at the doses of 0.5 mg/kg, 0.25 mg/kg and 0.125 mg/kg respectively. Formulation was prepared fresh daily.

Reference drug-Donepezil hydrochloride was suspended in 0.5% Na-CMC to get final concentration of 0.25 mg/mL (Dose: 2.5 mg/kg; Dose Volume: 10 mL/kg)

TBS buffer and 0.5% CMC were used as a vehicle for preparation of test item, recombinant Lectin having SEQ ID NO: 1 and reference item formulation, respectively Male Mus musculus (Swiss Albino) aged between 8-10 weeks procured from GENTOX Bio services Pvt Ltd, Hyderabad. Animals were classified into 6 groups and acclimatized for two weeks. Animals were identified by cage labelling and tail marking and were randomized on the basis of their body weight.

Healthy male Swiss albino mice (n=48) were selected and randomized based on body weight (n=8 per Group) into 6 groups as mentioned in table no-1. The control group G1 was treated intravenously with vehicle daily till the experiment completion. Group G2 was considered as negative control and treated with vehicle. Group G3 was treated with the standard compound Donepezil hydrochloride at the dose of 2.5 mg/kg, orally. Group G4, G5 &G6 animals were treated with the test item recombinant lectin protein having SEQ ID NO: 1 at the dose of 0.5 mg/kg, 0.25 mg/kg and 0.125 mg/kg, respectively. All the test items were given intravenously (i.v) daily at the dose volume of 5 ml/kg for 14 Days. The body weight was recorded daily throughout the experimental period. All animals were observed for clinical sign throughout the study. One hour after the last dose of test items (on Day 14) all the animals were injected with scopolamine hydrobromide at the dose of 2.5 mg/kg, i.p., except the normal control group G1. Passive avoidance test was conducted 30 min after scopolamine injection.

TABLE 9
Allocation of Animals

| Groups | Treatment | Dose and ROA | No. of animals |
|---|---|---|---|
| G1 | Normal Control + Vehicle TBS | 5 mL/kg, i.v. qdx14 | 8 |
| G2 | Scopolamine hydrobromide | 2.5 mg/kg, i.p. + | 8 |
| | (S) + Vehicle − TBS | 5 mL/kg, i.v. qdx14 | |
| G3 | S + Reference Compound | 2.5 mg/kg, i.p. + | 8 |
| | (Donepezil hydrochloride) | 2.5 mpk; p.o, 10 ml/kg. qdx14 | |
| G4 | S + SEQ ID NO: 1 | 2.5 mg/kg, i.p. + | 8 |
| | | 0.5 mg/kg, i.v, qdx14 | |
| G5 | S + SEQ ID NO: 1 | 2.5 mg/kg, i.p. + | 8 |
| | | 0.25 mg/kg, i.v., qdx14 | |
| G6 | S + SEQ ID NO: 1 | 2.5 mg/kg, i.p. + | 8 |
| | | 0.125 mg/kg, i.v., qdx14 | |

### Example 5a: Passive Avoidance Test

This learning and memory test were performed in two chambers, which has square boxes, identical size, juxtaposed as illuminated and dark. A lamp was placed above one chamber for illumination. Each test involved two separate trials, a training trial and a test trial.

For the training trial, the mice were initially placed in the illuminated chamber. When the mice entered the dark chamber, an electrical shock (0.5 mA) for 3 sec. was delivered through stainless steel rods. The latency times once the mice entered from light compartment to the dark compartment was recorded using in-build timer. A test trial was performed 24 h following the training trial, and latency times to re-enter the dark chamber was measured up to 5 min

TABLE 10
Mean Transfer Latency Time (Sec.)

| | | | % increase in |
|---|---|---|---|
| | | | Transfer |
| Groups | Mean | SEM | Latency Time |
| G1; Normal Control; Vehicle | 135.7 | 5.11 | NA |
| G2; Scopolamine hydrobromide | 56.93 | 5.12 | NA |
| (S) + Vehicle | | | |
| G3; S + Donepezil hydrochloride | 110.61 | 7.02 | 48.5 |
| G4; S + SEQ ID NO: 1; 0.5 mg/kg | 85.26 | 6.52 | 33.2 |
| G5; S + SEQ ID NO: 1; 0.25 mg/kg | 80.58 | 9.08 | 29.4 |
| G6; S + SEQ ID NO: 1; 0.125 mg/kg | 75.81 | 7.75 | 24.9 |

### Example 5b: Collection of Brain and Estimation of Brain Biomarkers

After passive avoidance test screening models of memory, the animals were sacrificed humanely. The whole brain was carefully removed from the skull and weighed. Brain was dissected into two portions

### One Portion of Brain:

10% w/v brain homogenate (100 mg/mL) was prepared by homogenizing it in ice-chilled phosphate buffer. The homogenate was subsequently centrifuged using a refrigerated centrifuge at 3000 rpm for 10 min, and the supernatant was separated and used for the biochemical estimations.

Following biomarkers was estimated using ELISA kit as per manufacturer's instruction.
Brain NGF (Nerve Growth Factor), CUSABIO, Catalog No. CSB-E04684m
Acetylcholinesterase, CUSABIO, Catalog No. CSB-E17521m
TNF alpha, CUSABIO, Catalog No. CSB-E04741m

TABLE 11
Effect of SEQ ID NO: 1 on brain
nerve growth factor (NGF) (pg/ml)

| Groups | Mean | SEM |
|---|---|---|
| G1; Normal Control; Vehicle | 74.22 | 3.29 |
| G2; Scopolamine hydrobromide | 34.49 | 2.38 |
| (S) + Vehicle | | |
| G3; S + Donepezil hydrochloride | 57 | 2.38 |
| G4; S + SEQ ID NO: 1; 0.5 mg/kg | 71.59 | 7.44 |
| G5; S + SEQ ID NO: 1; 0.25 mg/kg | 61.96 | 2.6 |
| G6; S + SEQ ID NO: 1; 0.125 mg/kg | 38.62 | 2.3 |

TABLE 12
Effect of SEQ ID NO: 1 on brain Acetylcholine esterase (mU/ml)

| Groups | Mean | SEM |
|---|---|---|
| G1; Normal Control; Vehicle | 4.19 | 0.29 |
| G2; Scopolamine hydrobromide | 6.74 | 0.36 |
| (S) + Vehicle | | |
| G3; S + Donepezil hydrochloride | 4.38 | 0.4 |
| G4; S + SEQ ID NO: 1; 0.5 mg/kg | 4.47 | 0.45 |
| G5; S + SEQ ID NO: 1; 0.25 mg/kg | 6.27 | 0.44 |
| G6; S + SEQ ID NO: 1; 0.125 mg/kg | 6.59 | 0.26 |

TABLE 13
Effect of SEQ ID NO: 1 on brain TNF-alpha (pg/ml)

| Groups | Mean | SEM |
|---|---|---|
| G1; Normal Control; Vehicle | 61.02 | 6.72 |
| G2; Scopolamine hydrobromide | 143.99 | 25.05 |
| (S) + Vehicle | | |
| G3; S + Donepezil hydrochloride | 155.81 | 14.99 |
| G4; S + SEQ ID NO: 1; 0.5 mg/kg | 121.73 | 23.65 |
| G5; S + SEQ ID NO: 1; 0.25 mg/kg | 67.65 | 5.91 |
| G6; S + SEQ ID NO: 1; 0.125 mg/kg | 79.53 | 11.63 |

### Example 5c: Histopathology

Mouse brains were collected after humane sacrifice and fixed in 10% neutral buffered formalin. Then, the brain tissue was trimmed, processed and embedded in paraffin. 5 Micron sections were prepared in slide as a specimen for hematoxylin-eosin (H&E) staining. The hippocampal lesions were assessed microscopically at 100× magnification.

### Result

Detailed histological investigation was performed H&E stained brain tissues. Scoring was given according to the neuronal damage. Histopathological evaluation results showed that no significant changes were observed in all the treatment groups as well as scopolamine injected group in cerebral cortex and hippocampus region except the CA3 region. The grading criteria for Histopathology score was: Normal or no injury—0; Rare neuronal injury (<5 clusters)—1; Occasional neuronal injury (5-15 clusters)—2; Frequent neuronal injury (>15 clusters)—3; Diffused neuronal injury-4.

TABLE 14
Histopathology (Mean score)

| Organs | Findings | G1 | G2 | G3 | G4 | G5 | G6 |
|---|---|---|---|---|---|---|---|
| Cerebral Cortex | Meninges | 0 | 0 | 0 | 0 | 0 | 0 |
| | Cortex | 0 | 0 | 0 | 0 | 0 | 0 |
| Hippocampus | CA1 | 0 | 0 | 0 | 0 | 0 | 0 |
| | CA3 | 0 | 2 | 1.33 | 0.67 | 0.6 | 1 |
| | DG | 0 | 0 | 0 | 0 | 0 | 0 |

We claim:

1. A recombinant lectin protein for treatment or prevention of neurodegenerative disease wherein the recombinant lectin protein is derived from Sclerotium rolfsii lectin.

2. The recombinant lectin protein as claimed in claim 1, wherein the recombinant lectin protein is selected from SEQ ID NO. 1, SEQ ID NO. 2, SEQ ID NO. 3 or amino acid sequence having at least 70% homology to SEQ ID NO. 4.

3. The recombinant lectin protein as claimed in claim 2, wherein the amino acid sequence has at least 75%, 80%, 90%, 95%, 96%, 97%, 98% or 99% homology to SEQ ID NO. 4.

4. The recombinant lectin protein as claimed in claim 1, wherein the neurodegenerative disease is selected from Alzheimer's disease, Parkinson's disease, dementia, cognitive disorder, symptoms related to dementia, Huntington's disease, prion diseases such as Creutzfeld-Jacob disease, Lewy Body disease, diffuse Lewy body disease (DLBD), polyglutamine (polyQ)-repeat diseases, cerebral degenerative diseases, spinal and bulbar muscular atrophy (SBMA), Ataxia, Pick's disease, primary progressive aphasia, multiple system atrophy, pantothenate kinase-associated neurodegeneration (PANK), spinal degenerative disease/motor neuron degenerative diseases, hippocampal sclerosis, corticobasal degeneration, Batten disease motor neuron disease like Amyotrophic lateral sclerosis (ALS, also termed Lou Gehrig's disease), primary lateral sclerosis (PLS), progressive bulbar palsy (PBP) a variant of ALS, Pseudo bulbar palsy and Hereditary spastic paraplegia.

5. The recombinant lectin protein as claimed in claim 4, wherein the neurodegenerative is selected from Alzheimer's disease, Parkinson's disease, dementia, cognitive disorder and symptoms related to dementia.

6. The recombinant lectin protein as claimed in any preceding claim, wherein the effective amount of recombinant lectin protein administered for the treatment or prevention of the neurodegenerative disease is in the range from 0.01 mg/kg to 1000 mg/kg body weight of the subject.

7. A pharmaceutical composition for the treatment or prevention of neurodegenerative disease comprising therapeutically effective amount of recombinant lectin protein derived from Sclerotium rolfsii lectin and a pharmaceutically acceptable excipient.

8. The pharmaceutical composition for the treatment or prevention of neurodegenerative as claimed in claim 7, wherein the neurodegenerative disease is selected from Alzheimer's disease, Parkinson's disease, dementia, cognitive disorder, and symptoms related to dementia.

9. A method of treatment or prevention of neurodegenerative disease in a subject, the method comprising administering a recombinant lectin protein recombinant lectin protein derived from Sclerotium rolfsii having sequence of SEQ ID NO. 1, SEQ ID NO. 2 or SEQ ID NO. 3 or the pharmaceutical composition of claim 7 to the subject.

10. The method of treatment or prevention of neurodegenerative disease in a subject as claimed in claim 11, wherein the amino acid sequence has at least 75%, 80%, 90%, 95%, 96%, 97%, 98% or 99% homology to SEQ ID NO. 4.

11. The method of treatment or prevention of neurodegenerative disease in a subject as claimed in claim 11, wherein the neurodegenerative disease is selected from Alzheimer's disease, Parkinson's disease, dementia, cognitive disorder, symptoms related to dementia, Huntington's disease, prion diseases such as Creutzfeld-Jacob disease, Lewy Body disease, diffuse Lewy body disease (DLBD), polyglutamine (polyQ)-repeat diseases, cerebral degenerative diseases, spinal and bulbar muscular atrophy (SBMA), Ataxia, Pick's disease, primary progressive aphasia, multiple system atrophy, pantothenate kinase-associated neurodegeneration (PANK), spinal degenerative disease/motor neuron degenerative diseases, hippocampal sclerosis, corticobasal degeneration, Batten disease motor neuron disease like Amyotrophic lateral sclerosis (ALS, also termed Lou Gehrig's disease), primary lateral sclerosis (PLS), progressive bulbar palsy (PBP) a variant of ALS, Pseudo bulbar palsy and Hereditary spastic paraplegia

12. The method of the treatment or prevention of neurodegenerative disease in a subject as claimed in claim 11, wherein the neurodegenerative is selected from Alzheimer's disease, Parkinson's disease, dementia, cognitive disorder and symptoms related to dementia.

13. The method of treatment of neurodegerative disease as claimed in claim 10 comprising administering recombinant lectin protein at a dose of 0.01 mg/kg to 1000 mg/kg.

14. Use of recombinant lectin protein in the manufacture of a medicament for the treatment or prevention of neurodegenerative disease wherein the recombinant lectin protein is derived from Sclerotium rolfsii lectin.

15. The recombinant lectin protein for use in the treatment or prevention of neurodegenerative disease as claimed in claim 20, wherein the amino acid sequence has at least 75%, 80%, 90%, 95%, 96%, 97%, 98% or 99% homology to SEQ ID NO. 4.

16. The recombinant lectin protein for use in the treatment or prevention of neurodegenerative disease as claimed in claim 20, wherein the neurodegenerative disease is selected from Alzheimer's disease, Parkinson's disease, dementia, cognitive disorder, symptoms related to dementia, Huntington's disease, prion diseases such as Creutzfeld-Jacob disease, Lewy Body disease, diffuse Lewy body disease (DLBD), polyglutamine (polyQ)-repeat diseases, cerebral degenerative diseases, spinal and bulbar muscular atrophy (SBMA), Ataxia, Pick's disease, primary progressive aphasia, multiple system atrophy, pantothenate kinase-associated neurodegeneration (PANK), spinal degenerative disease/motor neuron degenerative diseases, hippocampal sclerosis, corticobasal degeneration, Batten disease motor neuron disease like Amyotrophic lateral sclerosis (ALS, also termed Lou Gehrig's disease), primary lateral sclerosis (PLS), progressive bulbar palsy (PBP) a variant of ALS, Pseudo bulbar palsy and Hereditary spastic paraplegia.

17. The recombinant lectin protein for use in the treatment or prevention of neurodegenerative disease as claimed in claim 20, wherein the neurodegenerative is selected from Alzheimer's disease, Parkinson's disease, dementia, cognitive disorder and symptoms related to dementia

18. A method for inducing neuronal outgrowth in a subject affected by neurodegenerative disease, wherein the method comprises administration of an effective amount of recombinant lectin protein having sequence SEQ ID NO. 1, SEQ ID NO. 2 or SEQ ID NO. 3 to the subject, wherein the neurodegenerative disease is selected from Alzheimer's disease, Parkinson's disease, dementia, cognitive disorder and symptoms related to dementia.
